(19) United States
(12) Patent Application Publication
(10) Pub. No.: US 2026/0199274 A1
(43) Pub. Date: Jul. 16, 2026

(54) ACONITATE FOR THE TREATMENT OF A VIRAL LUNG INFECTION

(71) Applicants: UNIVERSITE DE TOURS, Tours (FR); INSTITUT NATIONAL DE LA SANTÉ ET DE LA RECHERCHE MÉDICALE, Paris (FR); CHRU DE TOURS, Tours (FR)

(72) Inventors: Mustapha SI-TAHAR, Tour (FR); Antoine Guillon, Veigne (FR)

(21) Appl. No.: 19/136,488

(22) PCT Filed: Dec. 14, 2023
(86) PCT No.: PCT/EP2023/085945

§ 371 Date: Jun. 6, 2025

(30) Foreign Application Priority Data
Dec. 16, 2022 (EP) 22306925.3

(51) Int. Cl. A61K 31/194; A61K 45/06; A61P 29/00; A61P 31/14; A61P 31/16

(52) U.S. Cl. CPC A61K 31/194; A61K 45/06; A61P 29/00; A61P 31/14; A61P 31/16

(57) ABSTRACT

The present invention relates to aconitate or a pharmaceutically acceptable salt thereof for use in a method of treatment of a viral lung infection and/or of an adverse immune response to a viral lung infection, the method comprising administering to a subject in need thereof an effective amount of aconitate or pharmaceutically acceptable salt thereof, or to a pharmaceutical composition comprising aconitate or a pharmaceutically acceptable salt thereof and a pharmaceutically acceptable support for use in a method as defined above.

### BACKGROUND

Lung infections can be either a persistent and pervasive burden, such as influenza infections, or a sudden worldwide disruptive outbreak, such as SARS-CoV-2. This recent pandemic has shed new light on the critical importance of viruses in respiratory infections.

Influenza virus causes significant morbi/mortality each year, since the 1918 pandemic, and influenza pathogenesis as well as anti-influenza therapeutic strategies have been extensively investigated. The pathophysiology of influenza lung infection is the result of two phenomena: (i) the intrinsic viral pathogenicity, linked to its tropism for the airway cells of the host, and (ii) the adverse immune response of the subject, which usually comprises a hyper-inflammatory immune response. Indeed, a robust host immune response is required for the viral clearance but the massive cellular recruitment and release of cytotoxic molecules lead to lung hyper-inflammation and can be associated with lung damage, morbidity and death.

Two classes of anti-influenza drugs have been discovered but only one (neuraminidase inhibitors) is currently recommended by the WHO [7]. The introduction of antiviral therapies against influenza such as the neuraminidase inhibitor Tamiflu® (oseltamivir) has made it possible to reduce the duration of flu symptoms for patient suffering from influenza throughout the world. However, some degree of skepticism is still present regarding the real efficacy of NA inhibitors, notably following the 2014's Cochrane clinical meta-analysis that reported only a minimal shortening of influenza symptoms in children and adults with uncomplicated influenza but not in hospitalized patients [8]. In particular, current antiviral therapies address the intrinsic viral pathogenicity, but have little or no effect with regard to the host hyper-inflammatory immune response to the viral infection.

It therefore remains essential to rapidly develop new strategies for treating a viral lung infection and/or an adverse immune response to a viral lung infection.

### BRIEF DESCRIPTION

The present invention is based on the inventors surprising finding that aconitate could modulate the immune response triggered by a lung viral infection. In particular, the inventors have shown that aconitate have anti-inflammatory properties potent enough to interrupt the inflammatory cascades in play during influenza and SARS-CoV-2 infection. In particular, aconitate exhibits an advantageous immunomodulatory activity in the lungs of subjects suffering from a viral infection caused by respiratory viruses such as an influenza virus or a coronavirus, and even more surprisingly, exhibits a strong antiviral activity on some respiratory viruses including influenza.

Accordingly, an embodiment E1 of the present disclosure relates to aconitate or a pharmaceutically acceptable salt thereof for use in a method of treatment of a viral lung infection and/or of an adverse immune response to a viral lung infection, the method comprising administering to a subject in need thereof an effective amount of aconitate or pharmaceutically acceptable salt thereof.

An embodiment E2 of the present disclosure relates to aconitate for use in a method according to embodiment E1, wherein the aconitate is cis-aconitate or trans-aconitate.

An embodiment E3 of the present disclosure relates to aconitate for use in a method according to any of embodiments E1 or E2, wherein the aconitate has immunomodulatory properties and/or antiviral properties, wherein:
the immunomodulatory properties of aconitate include the reduction or inhibition of the inflammation response to the viral lung infection;
the antiviral properties of aconitate include the inhibition of viral replication.

An embodiment E4 of the present disclosure relates to aconitate for use in a method according to any of embodiments E1 to E3, wherein the viral lung infection is caused by a respiratory virus preferably selected from an influenza virus, a coronavirus, a respiratory syncytial virus, a parainfluenza virus, a metapneumovirus, a rhinovirus, an adenovirus, a varicella-zoster virus, a cytomegalovirus, a paramyxovirus such as Nipah virus, and a bocavirus.

An embodiment E5 of the present disclosure relates to aconitate for use in a method according to any of embodiments E1 to E4, wherein the viral lung infection is caused by an influenza virus or a coronavirus.

An embodiment E6 of the present disclosure relates to aconitate for use in a method according to any of embodiments E1 to E5, wherein the method is applied to treat a viral lung infection.

An embodiment E7 of the present disclosure relates to aconitate for use in a method according to any of embodiments 1 to 5, wherein the method is applied to treat an adverse immune response to a viral lung infection in a subject with a lung viral infection.

An embodiment E8 of the present disclosure relates to aconitate for use in a method according to any of embodiments E1 to E5, wherein the method is applied to prevent a lung viral infection from escalating to an adverse immune response associated to a viral lung infection in a subject with a lung viral infection.

An embodiment E9 of the present disclosure relates to aconitate for use in a method according to any of embodiments E1 to E10, wherein the adverse immune response to viral lung infection is the adverse immune response to the lung viral infection is a hyper-inflammatory immune response, a dyspnea, a tachypnea, a pneumonia in particular an acute pneumonia, an exacerbation of a chronic respiratory disease such as asthma or chronic obstructive pulmonary disease, a sepsis, a septic shock, a cytokine storm or an acute respiratory distress syndrome (ARDS).

An embodiment E10 of the present disclosure relates to aconitate for use in a method according to embodiments E1 to E12, wherein aconitate is administered between 4 and 14 days post infection.

An embodiment E11 of the present disclosure relates to aconitate for use in a method according to any one of embodiments E1 to E10, wherein the subject is an animal, preferably a human, a domestic bird or a pig.

An embodiment E12 of the present disclosure relates to aconitate for use in a method according to any one of embodiments E1 to E11, wherein aconitate is used alone or in combination with one or more active substance(s) selected from the group consisting of antivirals, antibiotics, and/or antalgics.

An embodiment E13 of the present disclosure relates to aconitate for use in a method according to any of embodiments E1 to E12, wherein said treatment is for preventing the occurrence of a lung viral infection, the method comprising administering aconitate in a subject that is not infected with a respiratory virus.

An embodiment E14 of the present disclosure relates to aconitate for use in a method according to any of embodiments E1 to E13, wherein the composition is administered intrapulmonary, nasally, orally, enterally, intravenously, intramuscularly and subcutaneously.

Another aspect of the disclosure relates to a pharmaceutical composition comprising aconitate or a pharmaceutically acceptable salt thereof and a pharmaceutically acceptable support for use in a method as defined in any of embodiments E1 to E14.

### DETAILED DESCRIPTION

The inventors have presently shown that, surprisingly, in the context of lung cells infected with influenza virus, aconitate exerts an advantageous immunomodulatory activity, and, even more surprisingly, also exerts a strong antiviral activity. Indeed, the inventors have shown that aconitate has immunomodulatory (in particular downmodulatory) properties potent enough to interrupt the inflammatory cascades in play during influenza and coronavirus infection.

They have also demonstrated that aconitate strongly inhibited the viral polymerase of influenza and prevented the expression of viral mRNA and protein synthesis, and subsequently the influenza virus replication. This antiviral effect was observed in a broad spectrum of types (e.g. A, B, . . . ) and subtypes (e.g. H3N2, H1N1, . . . ) of influenza viruses. The inventors have thus demonstrated a direct inhibitory action of aconitate on the two components of the pathophysiology of influenza infection, namely the intrinsic viral pathogenicity and the hyper-inflammatory immune response associated to influenza infection.

Remarkably, the inventors have shown that aconitate treatment decreases IL-6 production in dose-dependent manner in bronchial epithelial cells stimulated with various kind of inflammatory agonists. Without wanting to be bound by any theory, the inventors believe that these findings strongly suggest that aconitate possess potent intrinsic downregulatory properties of therapeutic interest in a wide range of viral infections.

Importantly, the inventors have demonstrated the protective effects of aconitate not only in in vitro but also in vivo in a murine model and ex vivo in human lungs.

Thus, an aspect of the present disclosure relates to aconitate or a pharmaceutically acceptable salt thereof for use in a method of treatment of a viral lung infection and/or of an adverse immune response to a viral lung infection, the method comprising administering to a subject in need thereof an effective amount of aconitate or pharmaceutically acceptable salt thereof.

### Aconitate or Pharmaceutically Acceptable Salt Thereof

The aconitate may be cis-aconitate or trans-aconitate.

The aconitate is preferably cis-aconitate.

In the present disclosure, the term “pharmaceutically acceptable salt” denotes a salt of aconitate which has little or no undesired toxicological effect. The counter-ion may for example be selected from aluminum, arginine, benzathine, calcium, chloroprocaine, choline, diethanolamine, ethanolamine, ethylenediamine, lysine, magnesium, histidine, lithium, meglumine, potassium, procaine, sodium, triethylamine, and zinc, preferably is selected from sodium, and potassium.

In some embodiment, the pharmaceutically acceptable salt of aconitate is a sodium salt of cis-aconitate or trans-aconitate.

In some embodiment, the pharmaceutically acceptable salt of aconitate is a potassium salt of cis-aconitate or trans-aconitate.

In what follows, the expression “aconitate” and “aconitate or pharmaceutically acceptable salt thereof” are used interchangeably.

### Lung Viral Infection

As used in the present disclosure, the term “lung viral infection” relates to a condition characterized by (i) the proliferation of one or more virus in the lungs of the subject, and/or (ii) one or more adverse immune response associated to or induced by said viral infection in the lungs of the subject. A lung is a specific organ within the respiratory system. Humans typically have two lungs. According to the present disclosure, lung viral infections may affect other organs of the respiratory system selected from the group consisting of the pharynx, the larynx or the trachea.

### Adverse Immune Response

As used herein, the expression “adverse immune response”, or “host adverse immune response”, or “subject's adverse immune response” refers to a syndrome of physiologic, pathologic, and/or biochemical abnormalities triggered by a viral infection. The expression “adverse immune response” refers also to organ dysfunction due to dysregulated host response to infection than can be defined as “sepsis”.

Clinical manifestations of an adverse immune response to a lung viral infection usually include one or more of abnormal body temperature (typically characterized in human by a body temperature greater than 38° C. or lower than 36° C.), dyspnea, tachypnea (typically characterized in human by a respiratory rate greater than 20/min), bronchitis, pneumonia, sepsis-induced organ dysfunction, and acute respiratory distress syndrome (ARDS).

Biological manifestations usually include one or more of abnormal white blood cell count (typically characterized in human by a WBC count greater than 12 000/mm³ or lower than 4000/mm³ or greater than 10% immature bands), excessive release of systemic cytokines such as tumor necrosis factor (TNF), interleukin-1 (IL-1), interleukin-6 (IL-6), interleukin-8 (IL-8), hyper-inflammatory immune response, such as a cytokine storm or cytokine release syndrome, coagulopathy (consumption of coagulation factors, fibrinogen, and platelets), hypoxemia assessed by arterial blood gas analysis (typically characterized by a PaO2/FiO2 inferior or equal to 300 mmHg). Imaging manifestation usually includes the presence of pulmonary infiltrates on chest radiograph and/or CT scan.

In some embodiments, the lung viral infection induces or is capable of inducing an adverse immune response in the subject. In some embodiments, the adverse immune response to the lung viral infection is a hyper-inflammatory immune response, a dyspnea, a tachypnea, a pneumonia in particular an acute pneumonia, an exacerbation of a chronic respiratory disease such as asthma or chronic obstructive pulmonary disease, a sepsis, a septic shock, a cytokine storm or an acute respiratory distress syndrome (ARDS).

In some embodiments, the adverse immune response to the lung viral infection is pulmonary hypertension or hypotension.

In some embodiments, the adverse immune response to the lung viral infection is multi-organ failure. Multi-organ failure may comprise heart failure, liver failure, lung failure kidney failure, or gastrointestinal (GI) system failure.

In some embodiments, the lung viral infection and/or adverse immune response to the lung viral infection causes a viral respiratory disease in the subject, usually referred to as influenza (also referred to as “flu”), Coronavirus disease 2019 (COVID-19), severe acute respiratory syndrome (SARS), Middle East respiratory syndrome (MERS), or common cold.

In some embodiments, the lung viral infection and/or adverse immune response to the lung viral infection favors the occurrence of bacterial lung co-infection or superinfection.

### Immunomodulatory and Antiviral Properties

In some embodiments, aconitate for use according to the disclosure has immunomodulatory properties and/or antiviral properties, preferably wherein:
the immunomodulatory properties of aconitate include the reduction or inhibition of the inflammation response to the viral lung infection;
the antiviral properties of aconitate include the reduction or inhibition of viral replication.

### Immunomodulatory Properties

In some embodiment, aconitate has immunomodulatory properties.

In the present disclosure, the term “immunomodulatory medicament” or “immunomodulatory” refers to a substance that inhibits or reduces one or more adverse immune responses to a lung disease due to a virus in the subject, in particular a hyper-inflammatory immune response to the lung viral infection. The capacity of a substance to reduce an inflammatory response may for example be confirmed by measuring a diminution of the expression of some components of the inflammatory cascade such as IL-6, IL-8 or tumor necrosis factor-alpha (TNF-α) in the presence of that substance.

In some embodiments, the immunomodulatory properties of aconitate include inhibiting or reducing the lung immune inflammation response triggered by a virus.

In some embodiments, the aconitate for use according to the disclosure is for treating a lung viral infection or a pneumonia as an anti-inflammatory medicament, particularly for treating or preventing an adverse immune response to a lung viral infection.

In some embodiments, the aconitate for use according to the disclosure is for treating an adverse immune response to a lung virus infection selected from hyper-inflammatory immune response, selected from acute exacerbations of a chronic respiratory disease such as asthma or chronic obstructive pulmonary disease, pneumonia, sepsis, septic shock, cytokine storm and acute respiratory distress syndrome, in particular in moderate to advanced cases of said lung infection, in particular wherein said lung infection is respiratory viral infection caused by an influenza virus or a coronavirus such as SARS-CoV-2.

### Antiviral Properties

In some embodiments, aconitate has antiviral properties.

In the present disclosure, the term “antiviral medicament” or “antiviral” refers to a substance that inhibits or reduces virus replication in a subject.

In some embodiments, the antiviral properties of aconitate include inhibition of viral replication.

In some embodiments, the aconitate for use according to the disclosure treats a lung viral infection as an antiviral medicament, more particularly by disrupting the life cycle of the respiratory virus.

In some preferred embodiments, the aconitate for use according to the disclosure treats the respiratory viral infection both as an immunomodulatory and as an antiviral medicament.

### Respiratory Virus

In the present disclosure, the term “respiratory virus” refers to a virus having a tropism for the airway cells and in particular for the lungs of the subject, and able to cause an adverse immune response as described herein.

In some embodiments, the respiratory virus is selected from an influenza virus, a coronavirus, a respiratory syncytial virus, a parainfluenza virus, a metapneumovirus, a rhinovirus, an adenovirus, a varicella-zoster virus, a cytomegalovirus, a paramyxovirus such as a Nipah virus, and a bocavirus.

### Influenza Virus

In some embodiments, the respiratory virus is an influenza virus.

In some embodiment, the influenza virus is an Influenza A virus, an Influenza B virus, an Influenza C virus or an Influenza D virus.

In some embodiments, the influenza A virus is of serotype H1N1, H1N2, H2N2, H2N3, H3N1, H3N2, H3N8, H5N1, H5N2, H5N3, H5N6, H5N8, H5N9, H6N1, H6N2, H7N1, H7N2, H7N3, H7N4, H7N7, H7N9, H9N2, or H10N7.

In some embodiment, the influenza virus is an Influenza B virus.

In some embodiment, the Influenza B virus is of serotype Victoria or Yamagata.

In some embodiments, the influenza virus is an influenza virus usually causing human influenza, for example a H1N1, H2N2, H3N2, H5N1, H7N7, H1N2, H9N2, H7N2, H7N3, or H10N7 influenza virus.

In some embodiments, the influenza virus is an influenza virus usually causing avian influenza, for example a H5N1 or H7N9 influenza virus.

In some embodiments, the influenza virus is an influenza virus usually causing swine or pig influenza such as H1N1, H1N2, H2N1, H3N1, H3N2, H2N3, or influenza c virus.

In some embodiments, the influenza virus is an influenza virus usually causing equine influenza such as H7N7 or H3N8 influenza virus.

In some embodiments, the influenza virus is an influenza virus usually canine influenza such as H3N8 influenza virus.

In some embodiment, the aconitate for use according to the disclosure reduces or inhibits both the influenza virus replication and the immune inflammation response to an influenza virus infection.

### Coronavirus

In some embodiments, the respiratory virus is a coronavirus virus of Orthocoronavirinae subfamily, as a coronavirus.

In some embodiments the coronavirus is selected from severe acute respiratory syndrome coronavirus 2 (SARS-CoV-2), severe acute respiratory syndrome coronavirus (SARS-CoV), or Middle East respiratory syndrome coronavirus (MERS-CoV) or beta-coronavirus.

In some embodiments the coronavirus is SARS-CoV-2.

In some embodiments, the aconitate for use according to the disclosure reduces or inhibits at least the immune inflammation response to the coronavirus infection, and optionally also reduces the immune inflammation response to a coronavirus infection.

### Method of Treatment

As used in this document, the term “treatment” or “therapy” refers to any action which makes it possible to reduce or suppress the symptoms associated with a pathological condition. It comprises both a curative treatment and a prophylactic treatment for a disease.

A curative treatment is defined by a treatment resulting in a cure or a treatment which relieves, improves and/or eliminates, reduces and/or stabilizes the symptoms of a disease or the suffering that it causes. The term “curative treatment” may refer to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease. In particular, with reference to the treatment of a lung viral infection, the term “curative treatment” may refer to the inhibition of the viral infection and/or of the adverse immune reaction associated to said viral infection. A prophylactic treatment comprises both a treatment resulting in the prevention of a disease and a treatment which reduces and/or delays the incidence of a disease or the risk of it occurring. The terms “improve” and “reduce” include, but do not require complete recovery or complete prevention. The term “prophylactic treatment” may refer to one or more of preventing the disease; for example, preventing a disease, condition or disorder in an individual who is at risk of experiencing or displaying the pathology or symptomatology of the disease, condition or disorder (i.e., preventing the development of the pathology and/or symptomatology); and (2) reducing and/or delaying the incidence of a disease in an individual who is experiencing or displaying the pathology or symptomatology of the disease, condition or disorder (i.e., reversing the pathology and/or symptomatology) such as decreasing the severity of disease or reducing or alleviating one or more symptoms of the disease.

In the context of the present disclosure, the term “treatment” preferably may refer to the curative or prophylactic treatment of viral lung infection or to the curative or prophylactic treatment of a lung viral invention and/or of an adverse immune response to a lung viral infection as described herein. In particular, with reference to the method of the present disclosure, the term “prophylactic treatment” may refer to the prevention of the viral infection and/or of the adverse immune reaction to said viral infection.

The disclosure also provides the use of aconitate or a pharmaceutically acceptable salt thereof as described optionally in association with a pharmaceutically acceptable support and/or one or more active substance as described herein for the manufacture of a medicament for the treatment of a viral lung infection and/or of an adverse immune response to said viral lung infection, as described herein.

In another embodiment, the present disclosure provides a method of treatment of a viral lung infection and/or of an adverse immune response associated to a viral lung infection, the method comprising administering to a subject in need thereof an effective amount of aconitate or pharmaceutically acceptable salt thereof optionally in association with a pharmaceutically acceptable support and/or one or more active substance as described herein.

### Dose

The aconitate used according to the disclosure is administered to the patient at an effective dose. The term “effective” dose” or “therapeutically effective dose” as used herein refers to the amount required to observe a curative or prophylactic activity on the lung infection, and for example an amount required to observe an inhibition or a reduction of viral infection and/or to the adverse immune reaction to the lung viral infection. The amount of aconitate to be administered and the duration of the treatment are evaluated by those skilled in the art according to criteria such as the physiological condition of the subject to be treated, the nature of the lung infection or lung hyperinflammation response to be treated, and the administration route used. The aconitate used according to the disclosure can be administered in the form of a single dose or multiple doses.

In some embodiment, the aconitate for use according to the present disclosure is administered to said subject in a therapeutically effective dose, for example at a dose to reach a concentration of about 0.3 to about 10 mM at the site of treatment (e.g. the respiratory tract). Nevertheless, this exemplary dose can vary within wide limits and is to be suited to the individual conditions in each individual case.

### Patient Selection

The subject to be treated, or patient, is an animal, preferably a mammal.

According to one preferred embodiment, the subject to be treated is a human, preferably an adult. In some embodiments, the subject is an aged human patient, in particular being more than 50, 60, 70, 80, 90 years old, more particularly being more than 65 years old. In some embodiments, the subject is a child, in particular a child being less than 2, 5, 7 or 10 years old.

In some embodiment, the subject to be treated is a subject vulnerable to lung viral infection. As used herein “vulnerable” denotes individuals that may encounter difficulty in protecting themselves are therefore at greater risk to suffers disproportionately from a lung viral infection and its complications. Subjects vulnerable to lung viral infection include but are not limited to aged patients, in particular aged patents of 65 years old or more, children in particular children of 2 years or less, and pregnant women.

According to one embodiment, the subject to be treated is an animal other than human, preferably a domestic animal selected from the group consisting of a bird, a dog, a cat, a horse, a cow, a sheep, a pig and a non-human primate.

The domestic bird is for example a chicken, a duck, a goose or a turkey.

In some embodiments, the subject is an animal, preferably a human or a domestic bird.

### The Subject to be Treated

### Subject to be Treated

The method of the present disclosure may be applied to a subject prior or after a viral lung infection, prior or after the appearance of an adverse immune response to said viral infection.

The method of the present disclosure can thus be applied to a subject that is infected or uninfected, i.e. at different levels of severity of the viral infection as described herein.

In some embodiments, the subject is at risk of developing an adverse immune response to the viral lung infection, for example to develop a pneumonia.

In some embodiments, the subject at risk of developing an adverse immune response to the viral lung infection is an aged human patient, preferably a human patient being more than 50, 60, 70, 80, 90 years old.

The subject is Infected

In some embodiments, the method of the disclosure is applied to a subject infected, i.e. having a viral lung infection.

In some embodiments, the method of the disclosure is applied to treat the viral lung infection.

In some embodiments, the method of the disclosure is applied to prevent the subject from developing an adverse immune response to the viral lung infection.

In some embodiments, the method of the disclosure is applied to prevent the subject from developing a hyper-inflammatory immune response, a dyspnea, a tachypnea, a pneumonia in particular an acute pneumonia, an exacerbation of a chronic respiratory disease such as asthma or chronic obstructive pulmonary disease, a sepsis, a septic shock, a cytokine storm or an acute respiratory distress syndrome (ARDS).

In some embodiments, the method of the disclosure is applied to prevent the lung viral infection from escalating to an adverse immune response.

### The Subject is Infected and Presents an Adverse Immune Reaction to the Infection

In some embodiments, the method of the disclosure is applied to treat the adverse immune response to a viral lung infection.

### The Subject is Uninfected.

In some embodiments, the method of the disclosure is applied to a subject uninfected with a viral infection.

In some embodiments, the method of the disclosure is applied to treat a lung viral infection and/or an adverse immune response to the viral lung infection.

### Severity/Stage of the Viral Infection

The method can be applied at various stages of the lung infection.

The inventors have shown that, surprisingly, aconitate was effective even when administered at a distance from the infection (4 days) while oseltamivir was ineffective.

This result strongly suggests that aconitate can treat an advanced stage lung infection. As used herein, “advanced stage lung infection”, refers to patient with lung infection that needs oxygen therapy. This is particularly advantageous because current antiviral treatments, such as Tamiflu®, are ineffective alone on advanced infections.

In some embodiments, the treatment according to the disclosure is for treating a subject at an advanced stage of the lung viral infection.

In these embodiments, the aconitate for use according to the disclosure is preferably administered to the subject at between 4 to 14 days post infection, preferably at between 5 to 11 days post infection, more preferably at 7 to 11 days post infection. In some embodiments, the aconitate for use according to the disclosure is preferably administered to the subject at between 4 to 11 days post infection, more preferably 4 to 7 days post infection.

In some other embodiments, the treatment according to the disclosure is for treating a subject at an early stage of the lung infection.

In these embodiments, the aconitate for use according to the disclosure is preferably administered to the subject immediately, one hour, 6 hours, 12 hours, 1 day, 2 days, 3 days post infection, between 8 to 24 hours post infection, more preferably between 10 to 20 hours post infection, even more preferably at 12 to 16 hours post infection.

In some embodiments, the method according to the disclosure is applied for preventing a lung viral infection from escalating to an adverse immune response associated to a viral lung infection in a subject that is suffering from a lung viral infection.

Without wanting to be bound by any theory, the inventors believe aconitate is probably most effective when given as soon as possible. Yet, as mentioned above the inventors have found that quite surprisingly, it is still effective 4 days after infection, which, from a practical and/or clinical point of view, is a significant advantage over the existing antiviral treatments such as Tamiflu® (oseltamivir).

The method of the present disclosure can be applied to subjects at various stages of the viral infection, i.e. to subjects presenting a variety of severity of viral lung infection and/or adverse immune response to lung viral infection.

The severity of the subject state may be assessed by a score according to the following ordinal scale where increasing numbers denote increased severity:

| Score | Descriptor | Subject state | |
|---|---|---|---|
| 0 | No clinical or virological | Uninfected | Uninfected/ |
| | evidence of infection | | Ambulatory |
| 1 | Not hospitalized, no limitation | Infected | |
| | of subject's activities | (Ambulatory) | |
| 2 | Not hospitalized, limitation | | |
| | of subject activities and/or | | |
| | requiring home oxygen | | |
| 3 | Subject is hospitalized but does | Infected - | Hospitalized |
| | not require oxygen therapy - | Hospitalized | but without |
| | requiring ongoing care | (Mild | ventilatory |
| 4 | Subject is hospitalized and | disease) | support |
| | requires supplemental oxygen | | |
| 5 | Subject is hospitalized and | Infected - | |
| | receives supplemental oxygen | Hospitalized | |
| 6 | Subject is hospitalized and | (severe | Hospitalized |
| | receives oxygen therapy that | disease) | with |
| | is non-invasive mechanical | | ventilatory |
| | ventilation or high flow | | support |
| | oxygen devices | | |
| 7 | Subject is hospitalized and | | |
| | receives invasive mechanical | | |
| | ventilation and/or additional | | |
| | organ support as vasopressors, | | |
| | renal replacement therapy, | | |
| | and/or extracorporeal | | |
| | membrane oxygenation | | |

In some embodiment, the subject has a severity assessed as uninfected/ambulatory.

In some embodiment, the subject has a severity assessed as hospitalized but without ventilatory support.

In some embodiment, the subject has a severity assessed as hospitalized with ventilatory support.

### Combination Therapy

The aconitate used according to the disclosure can be used as a sole active ingredient or in combination with one or more active substances. The aconitate and said active substance(s) can be administered simultaneously or sequentially.

In the present disclosure, the term “administering” means administration of a sole therapeutic agent or in combination with another therapeutic agent.

According to one embodiment, the aconitate is used in combination with one or more active substance selected from the group consisting of antivirals, antibiotics, and/or antalgics.

In some embodiments, the aconitate is used in combination with one or more antivirals, in particular antivirals of standard therapy of respiratory system viral infections, such as a neuramidase inhibitor (e.g. oseltamivir (Tamiflu®), zanamivir (Relenza®), peramivir (Rapivab®), favipiravir, remdesivir, ribavirin, interferon alpha 2a or 2b, molnupiravir, sotrovimab, casirivimab/imdevimab, baloxavir marboxil (Xofluza®)).

In some embodiments, the aconitate is used in combination with one or more antibiotics used in the treatment of bacterial co-infection, in particular antibiotics of standard antibiotherapy such as penicillins, cephalosporins, fluoroquinolones, aminoglycosides, carbapenems, and macrolides.

In some embodiments, the aconitate is used in combination with one or more antalgics such as acetaminophen (paracetamol), nefopam, tramadol, and opioids.

The aconitate used according to the disclosure may be administered via any known administration route, including intrapulmonary, systemically (parenterally, intravenously, etc.), orally, rectally, topically or subcutaneously.

### Routes of Administration

In some preferred embodiments, the aconitate used according to the disclosure is administered intrapulmonary, nasally, orally, enterally, intravenously, intramuscularly and subcutaneously.

In some preferred embodiments, the aconitate used according to the disclosure is administered intrapulmonary.

As used herein, “intrapulmonary”, refers to an administration route allowing to deliver the aconitate to the lungs and/or bronchi, where it particularly concentrates at the alveolar and/or bronchial epithelium.

In some embodiments, the aconitate for use according to the disclosure is preferably administered as an aerosol of a powder or aqueous solution or aqueous suspension, in particular using a nebulizer or a dry powder inhaler.

In some embodiments, the aconitate for use according to the disclosure is administered as an aerosol of a powder or aqueous solution or aqueous suspension in subject spontaneously breathing or receiving supplemental oxygen (including high oxygen devices) or being assisted by mechanical ventilation (non-invasive or invasive).

The term “aerosol”, as used in the present disclosure, refers to a dispersion of solid particles or liquid droplets in a gas adapted for targeting the lower airway passages, and preferably the lungs. A nebulizer is defined as a device capable of aerosolizing a liquid material (solution or dispersion) in the form of inhalable liquid droplets. The nebulizer allows the administration of said composition by means of a mask or a tip disposed on the mouth and/or the nose of the subject.

### Administration Regimen

In some embodiment, the aconitate for use according to the disclosure and optionally the one or additional active substance(s) as described herein is or are administered as a single dose, or in a fractionated dose regimen, simultaneously, separately, or sequentially.

In some embodiment, the aconitate for use according to the disclosure and optionally the one or additional active substance(s) as described herein is or are administered to the subject in a fractionated dose regimen.

In some embodiments, the fractionated dose regimen as described herein comprises 2 to 10 fractionated doses.

In a preferred embodiment, the fractionated dose regimen as described herein is administered once daily or once every two days.

In an embodiment, the fractionated doses as described herein are administered with a time lapse between two fractionated doses comprised between 4 h and 48 h, preferably between 4 h and 12 h, more preferably between 4 h and 10 h, for example with a time lapse of 6 hours.

### Pharmaceutical Composition

When employed as pharmaceutical, the aconitate for use as provided herein can be administered in the form of pharmaceutical composition.

The pharmaceutical composition for use according to the disclosure typically comprise aconitate and a pharmaceutically acceptable support, for use as described herein.

In the context of the disclosure, the term “pharmaceutically acceptable support denotes substances such as excipients, carriers, adjuvants, buffers or the like which are conventionally used, in combination with the active ingredient(s), for the preparation of a medicament. The choice of such supports depends essentially on the route of administration envisaged. Pharmaceutically acceptable supports include diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO₂), solvents/co-solvents (e.g. aqueous vehicle, Propylene glycol, glycerol), buffering agents (e.g. citrate, gluconates, lactates), preservatives (e.g. Na benzoate, parabens (Me, Pr and Bu), BKC), anti-oxidants (e.g. BHT, BHA, Ascorbic acid), wetting agents (e.g. polysorbates, sorbitan thickening esters), agents (e.g. methylcellulose or hydroxyethylcellulose), sweetening agents (e.g. sorbitol, saccharin, aspartame, acesulfame), humectants (e.g. propylene, glycol, glycerol, sorbitol). Other suitable pharmaceutically acceptable supports are inter alia described in Remington's Pharmaceutical Sciences, 15^{th} Ed., Mack Publishing Co., New Jersey (1991) and Bauer et ak, Pharmazeutische Technologic, 5^{th} Ed., Govi-Verlag Frankfurt (1997). The person skilled in the art knows will readily be able to choose suitable pharmaceutically acceptable supports, depending, e.g., on the formulation and administration route of the pharmaceutical composition.

In some embodiments, the aconitate may be in encapsulated form, by being, for example, introduced into microspheres or microcapsules which are reservoirs consisting of a core of active ingredient Surrounded by a membrane of coating material. The polymers forming the coating material may be of natural origin (gelatin, chitosan, etc.), semisynthetic origin (cellulose derivatives, etc.) or synthetic origin, such as the lactic and glycolic acid copolymers commonly used. The compounds of the disclosure may also be encapsulated in nanoparticles, which are colloidal systems of which the size is between 10 and 1000 nm, based on biodegradable polymers, or on lipids capable of retaining one or more active molecules by sequestration and/or adsorption.

The pharmaceutical composition according to the disclosure preferably comprises an amount of aconitate of between 5 μg and 1000 mg, preferably between 1 and 500 mg, preferably between 5 and 100 mg.

The ratio between the amounts by weight of compound according to the disclosure and of pharmaceutically accept able support is between 5/95 and 95/5, preferably between 20/80 and 80/20.

The pharmaceutical composition according to the disclosure may for example be formulated as a tablet, capsule, granule, powder, sachet, reconstitutable powder, dry powder inhaler and/or chewable. Such solid formulations may comprise excipients and other ingredients in suitable amounts. Such solid formulations may contain e.g. cellulose, cellulose microcrystalline, polyvidone, magnesium stearate and the like.

In some preferred embodiments, the pharmaceutical composition is for inhalation. In this case, the dosage can preferably be reduced because of the application of the drug directly to the site of action, i.e. the lungs.

The present disclosure also relates to a method for treating a lung infection, comprising the administration, to a subject, of an effective amount of aconitate and/or of a pharmaceutical composition containing the same.

A subject of the present disclosure is also the use of aconitate in the context of the preparation of a pharmaceutical composition intended for the treatment of a lung infection.

Other aspects and advantages of the present disclosure will emerge upon reading the examples which follow, which should be considered to be non-limiting illustrations.

### LEGENDS OF THE FIGURES

FIG. 1. Assessment of anti-influenza virus activity of metabolites derived from glycolysis or TCA cycle.

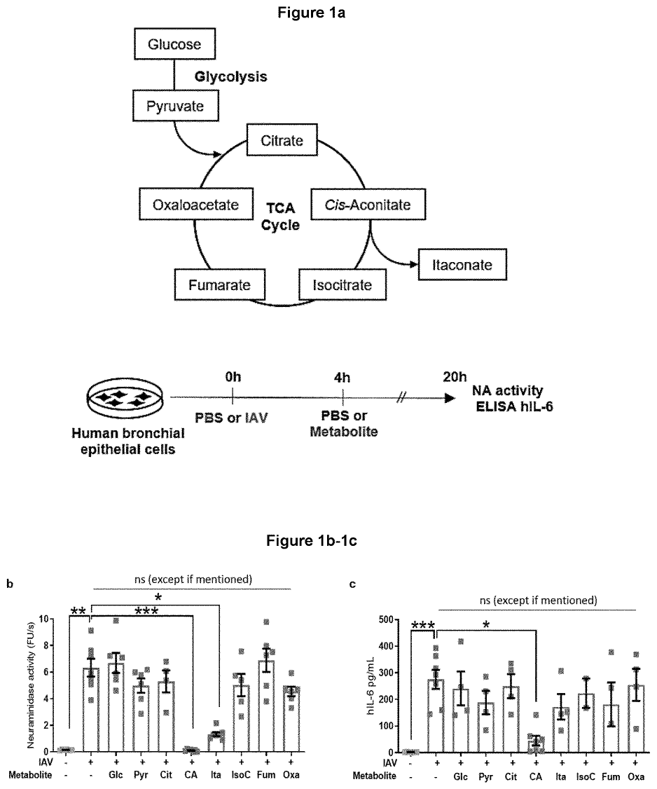

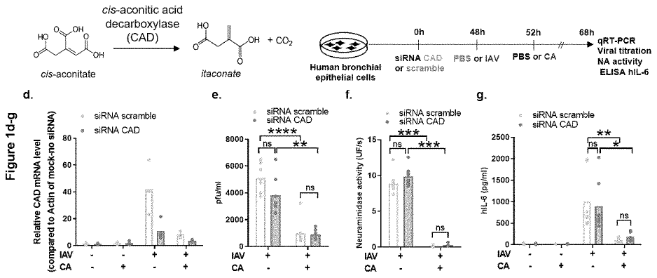

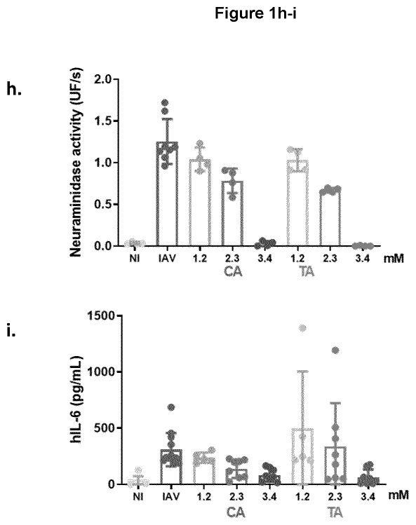

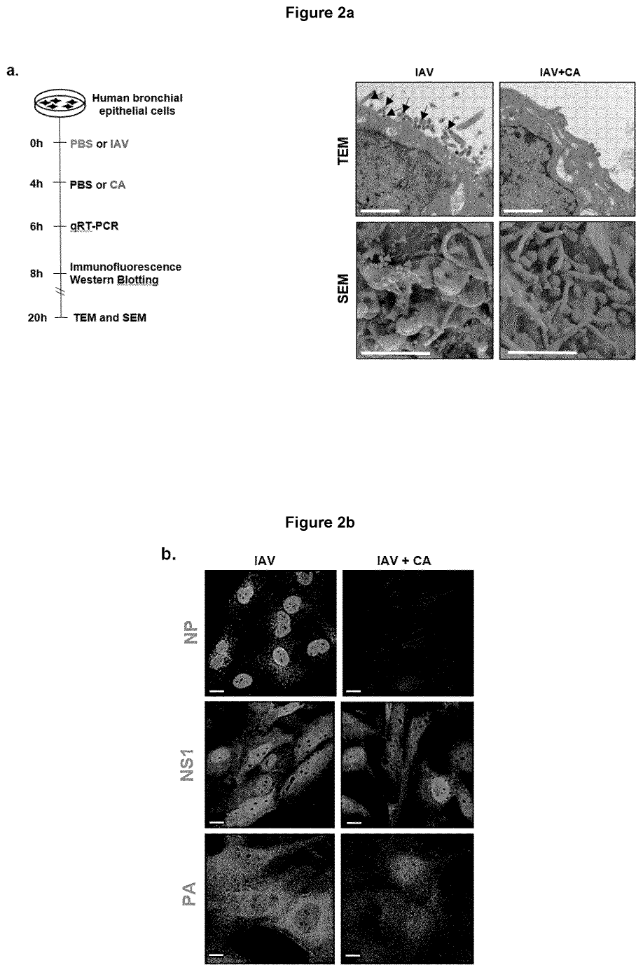

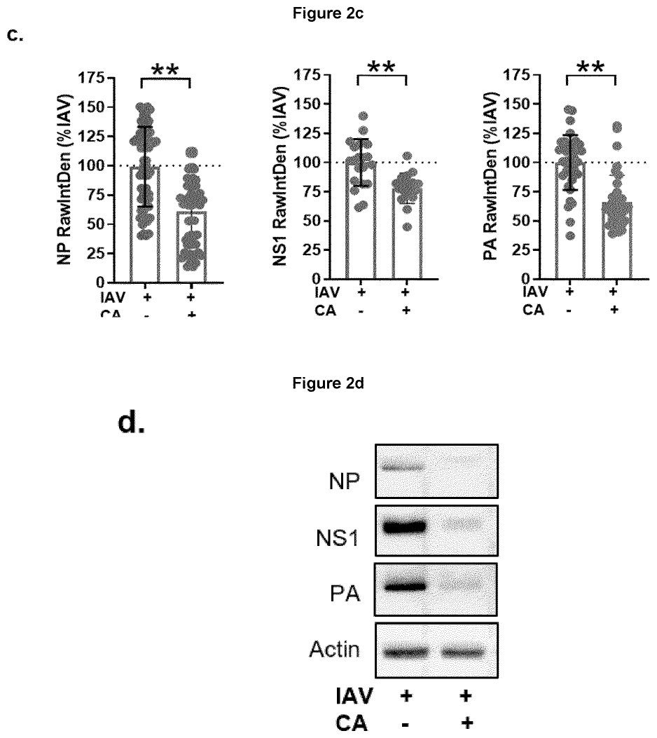

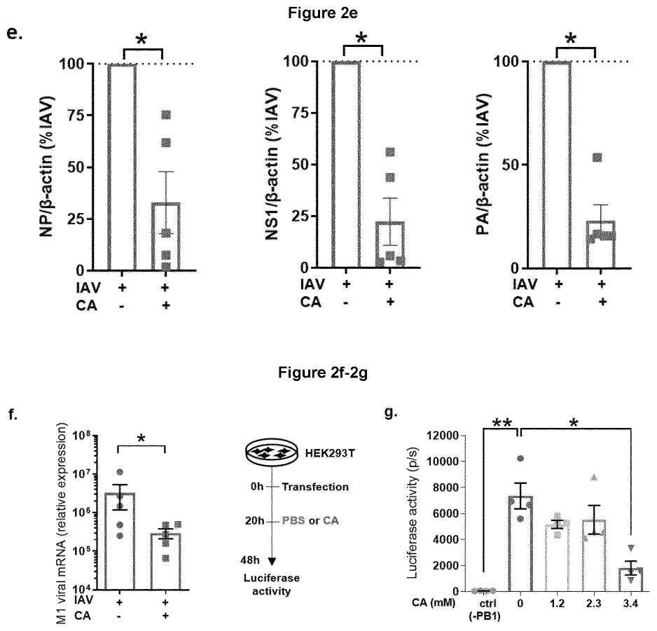

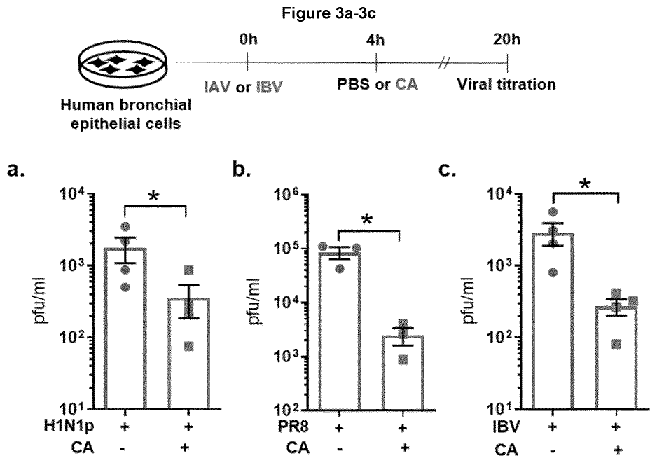

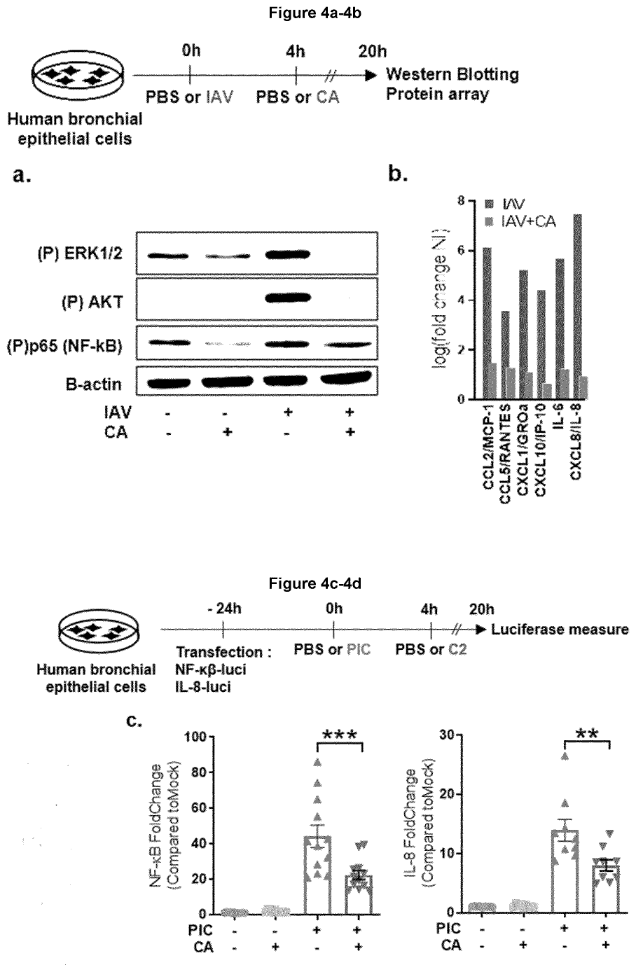

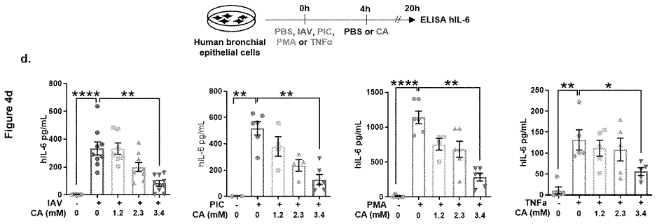

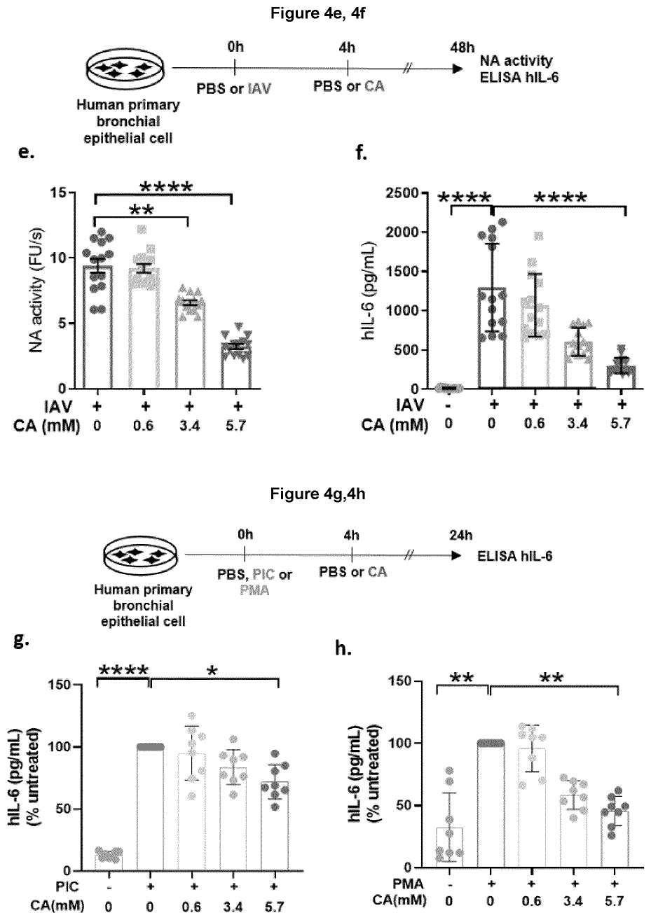

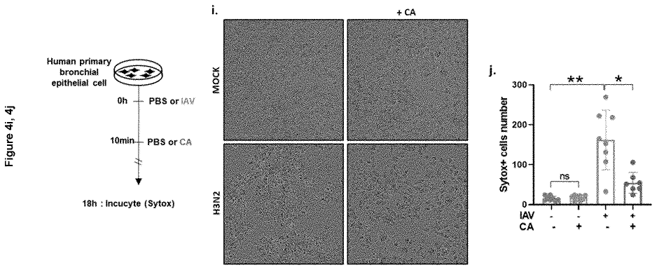

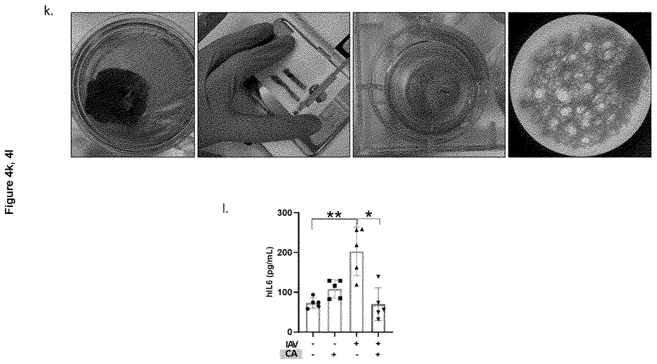

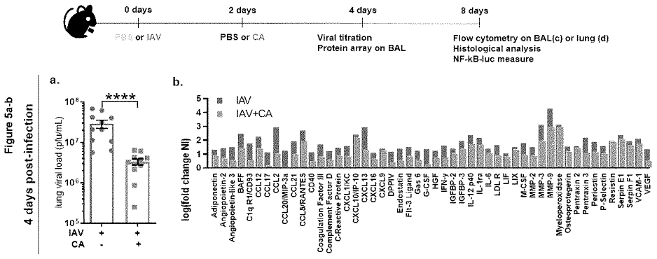

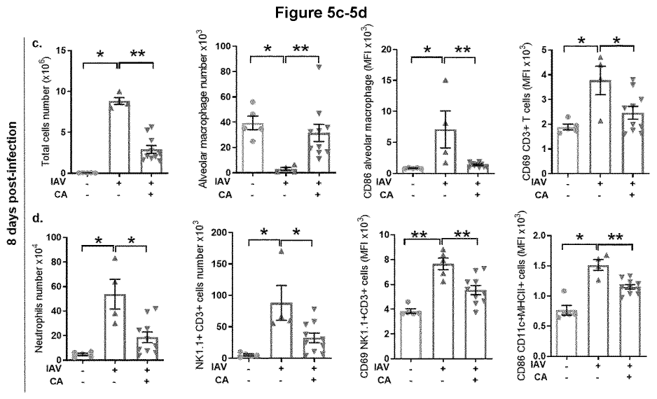

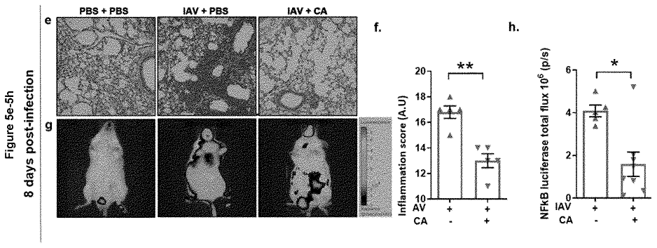

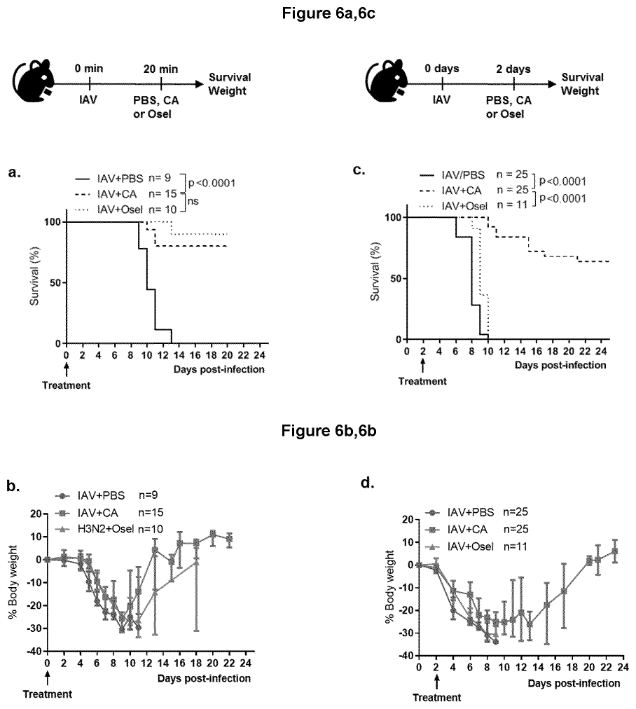

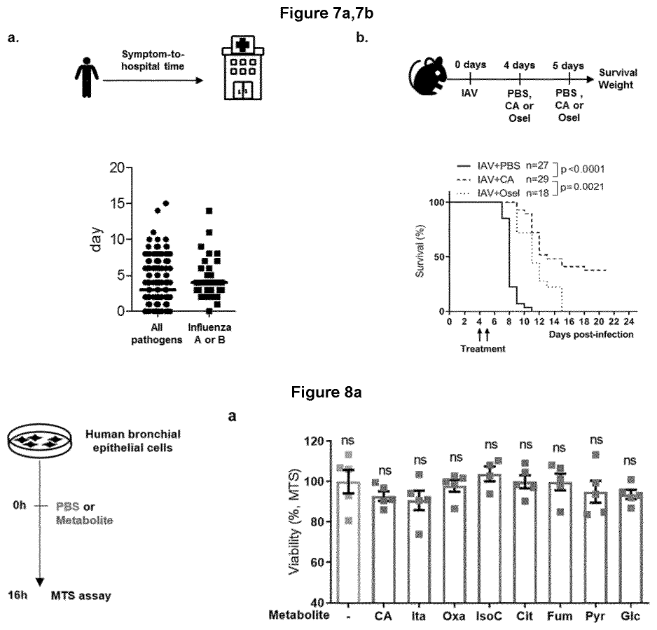

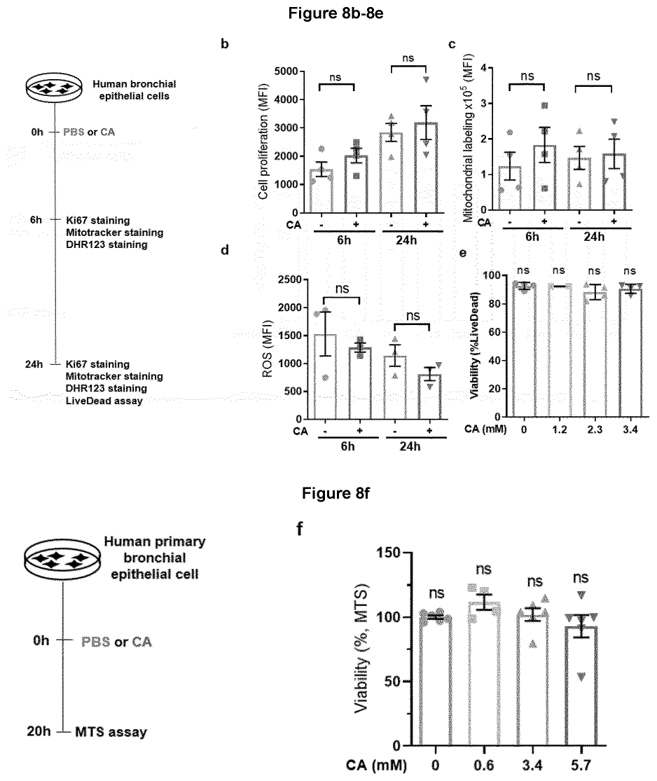

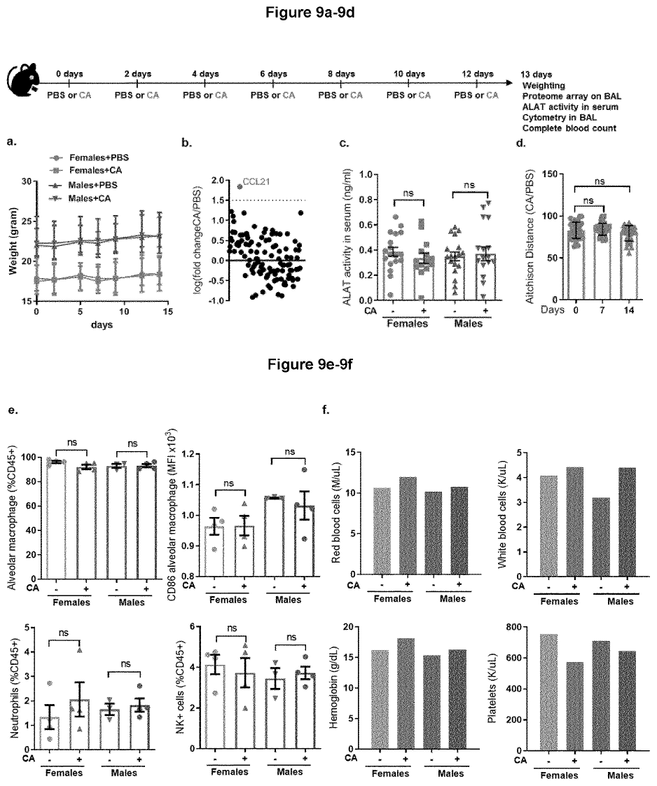

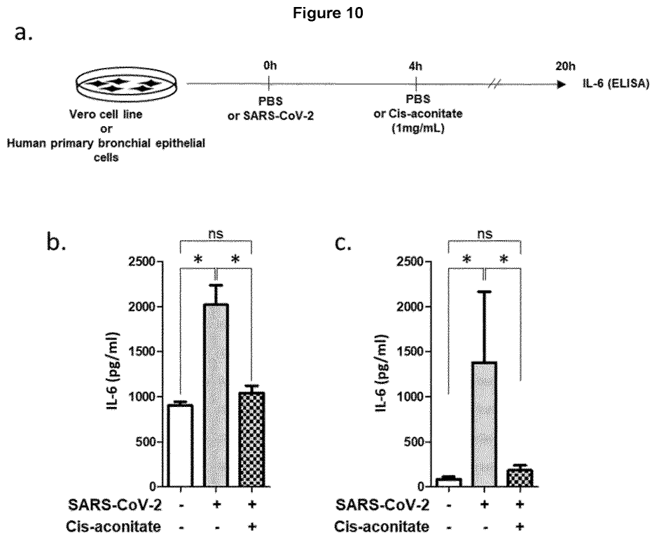

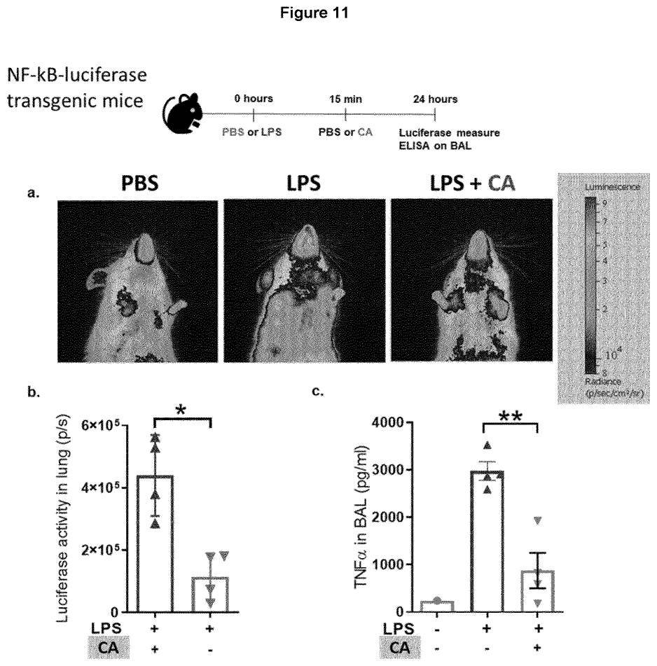

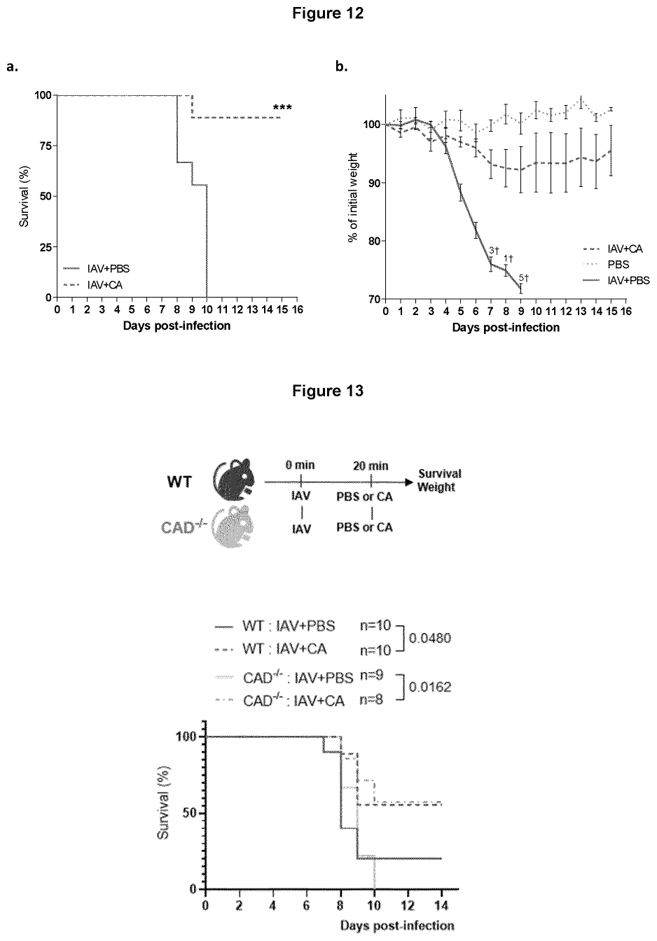

(FIG. 1a) Tested metabolites derived from glycolysis or TCA cycle. Bronchial epithelial (BEAS-2B) cells were infected with A/Scotland/20/74 (H3N2) virus at MOI=1 (IAV), or not (PBS), for 4 h and treated (Metabolite) or not (PBS) with 3.4 mM of TCA cycle- or glycolysis metabolites: cis-aconitate (CA), itaconate (Ita), oxaloacetate (Oxa), isocitrate (IsoC), citrate (Cit), fumarate (Fum), pyruvate (Pyr) or glucose (Glc) for 16 h. (FIG. 1b) The production of viral particles assessed by neuraminidase activity assay. (FIG. 1c) hIL-6 production in cell supernatant measured by ELISA. BEAS-2B cells were transfected with cis-aconitate decarboxylase (CAD) or control (scramble) siRNA. 48 h post-transfection, cells were infected or not with A/Scotland/20/74 (H3N2) virus (IAV) at MOI=1 for 4 h, and treated or not with 3.4 mM of cis-aconitate (CA) for 16 h. (FIG. 1d) gene knockdown was evaluated by RT-qPCR. (FIG. 1e, FIG. 1f) production of viral particles was assessed by plaque assay (FIG. 1e) and neuraminidase activity assay (f). (FIG. 1g) hIL-6 production in cell supernatant was measured by ELISA. All data are represented as the mean±SEM and are cumulative of 7 (FIG. 1b) or 4 (FIG. 1c and FIG. 1e-g) independent experiments. Statistical analysis was performed using the Kruskal-Wallis test with Dunn's multiple comparison test (FIG. 1a-g). Trans-aconitic acid (TA) also exhibited similar antiviral and anti-inflammatory activities, indicating that both isomers of aconitic acid possess anti-influenza properties (FIG. 1h-i).

FIG. 2: anti-influenza virus properties of cis-aconitate are mediated through inhibition of viral polymerase activity.

(FIG. 2a) Left: Human bronchial epithelial BEAS-2B cells were infected with A/Scotland/20/74 (H3N2) virus at MOI=1 or 5 (a) for 4 h, then washed and treated (CA) or not (PBS) with 3.4 mM or the indicated concentrations of cis-aconitate. Right. Representative images of 2 distinct transmission electron microscopy (TEM) analysis (upper panel) or 2 scanning electron microscope (SEM) experiments (lower panel). IAV particles budding from bronchial epithelial cells at 20 h pi is indicated by the arrows (scale bar: 1 μm). (FIG. 2b-e) At 8 h pi, viral protein expression and trafficking were analyzed by confocal microscopy and Western blotting to detect the viral NP, NS1, and PA proteins using specific antibodies. (FIG. 2b) Viral proteins are indicated in gray (scale bar=20 UM). (FIG. 2c) The value raw integrated density (RawIntDen, which is the sum of all pixel values in the ROI (region of interest)) was further measured and normalized on the mean of IAV condition of each experiment. (FIG. 2d, FIG. 2e) Viral protein expression assessed by Western blotting. Relative protein quantification was obtained by normalization over the mean value of “IAV condition” samples; (β-actin was used as a loading control) (FIG. 2f). At 6 h pi, IAV transcription was assessed by RT-qPCR to quantify the M1 viral mRNA. (FIG. 2g) Minigenome assay was performed on HEK-293T cells to test the effect of cis-aconitate on viral polymerase activity. 293T cells were transfected with pRF483-PA-RT, pRF483-PB2-RT, pRF483-NP-RT, pRF483-PB1-RT and the reporter plasmid pPoll-WSN-NA-firefly luciferase. 20 h post-transfection, cells were treated with 0, 1.2, 2.3 or 3.4 mM of cis-aconitate (CA). Luciferase activity was measured at 48 h post-transfection. results are expressed as the mean±SEM of 3 (FIG. 2a, 2b, 2c), 4 (FIG. 2d, 2e, 2f) or 5 (FIG. 2f) independent experiments. Statistical analysis was performed using Kruskal-Wallis test with Dunn's multiple comparison test (FIG. 2g), Mann-Whitney test (FIG. 2c), or Wilcoxon matched-pairs signed rank test (FIG. 2e, 2f).

FIG. 3. Cis-aconitate blocks the multiplication of different types and subtypes of influenza viruses.

Human bronchial epithelial BEAS-2B cells were infected with influenza A/pandemic/2009 H1N1 (H1N1p) strain (FIG. 3a), A/Puerto Rico/8/1934 H1N1 (PR8) virus (FIG. 3b) or influenza B Yamagata (B/Paris/234/2013) virus (IBV) (FIG. 3c). 4 h post-infection, cells were washed and treated or not for 16 h with 3.4 mM of cis-aconitate (CA). A Plaque-Forming Units assay was used to quantify the production of infectious viral particles in the cell supernatants. Data are represented as the mean±SEM of 3 (FIG. 3b) or 4 (FIG. 3a, 3c) independent experiments. Statistical analysis was performed using ratio paired t-test.

FIG. 4. Cis-aconitate reduces pro-inflammatory responses and signaling in lung epithelial cells.

(FIG. 4a-b) Human bronchial epithelial BEAS-2B cells were infected or not with A/Scotland/20/74 (H3N2) virus at MOI=1 for 4 h, and subsequently treated or not with 3.4 mM of cis-aconitate (CA) for 20 h. (FIG. 4a) Western blotting was used to detect the phosphorylated form (P) of ERK1/2, AKT and p65 proteins (β-actin was used as a loading control). (FIG. 4b) Levels of 6 immune mediators in cell supernatants were determined using a specific protein-array. Green bars represent the secretion of mediators induced by IAV (expressed as the fold change over the non-infected (NI) condition). Blue bars represent the secretion of mediators induced by IAV+CA in fold changes over the NI condition. (FIG. 4c) BEAS-2B cells were transfected with distinct plasmids, i.e. NF-κB-, IL-8-, ISG54- or ISG56-luciferase. 24 h post-transfection, cells were stimulated or not (grey) with 2 μg/mL of Poly (I:C) (PIC) for 4 h, and treated with 3.4 mM of cis-aconitate (CA) for 16 h. Levels of luciferase were measured 24 h post stimulation. (FIG. 4d) BEAS-2B cells were stimulated or not (PBS) with IAV at MOI=1 (IAV), 2 μg/mL of Poly (I:C) (PIC), 50 nM of Phorbol 12-myristate 13-acetate (PMA) or 20 ng/ml of Tumor necrosis factor alpha (TNFα) for 4 h, and treated with increasing doses of cis-aconitate (CA) for 16 h. Levels of IL-6 were measured by ELISA in the cell supernatants. Data are represented as the mean±SEM of at least 5 independent experiments. Statistical analysis was performed using the Ordinary two-way ANOVA Turkey's multiple comparisons test, with individual variances computed for each comparison (FIG. 4c), Kruskal-Wallis (FIG. 4d: IAV, PIC and PMA) or Friedman (FIG. 4d: TNFα) with Dunn's multiple comparison test. (FIG. 4e-h). Cis-aconitate has antiviral and anti-inflammatory properties in human primary bronchial epithelial cells (PBEC). (FIG. 4e-f) PBEC in liquid culture were infected with A/Scotland/20/74 (H3N2) virus at MOI=1 for 4 h, and subsequently treated or not with different concentration of cis-aconitate (CA) for 44 h. At 48 h pi, neuraminidase activity (FIG. 4e) and hIL-6 (FIG. 4f) were measured in cell supernatants to assess the production viral particles and pro-inflammatory cytokine release, respectively. (FIG. 4g-h) PBEC in liquid culture were incubated or not with poly(I:C) or PMA for 4 h, and subsequently treated or not with different concentrations of cis-aconitate (CA) for 20 h. Levels of IL-6 were further measured by ELISA. (FIG. 4i-j) PBECs in liquid culture were infected with virus A/Scotland/20/74 (H3N2) at MOI=1, then treated or not with 3.4 mM cis-aconitate (CA). SYTOX™ labelling was monitored for 18 hours. Representative images (FIG. 4a) and quantification of labeling (FIG. 4b) at 18 h pi were used to assess cell death. PBEC were obtained from 4 independent patients. (FIG. 4k-l) Human precision-cut lung slices (PCLS) were performed on lung tissues collected from patients who underwent thoracic surgery (first image, FIG. 4k). Lung explants were sliced into 400 μm thin slices (second image, FIG. 4k). Individualized PCLS were placed in air-liquid interface (third and fourth image, FIG. 4k). PCLS were infected with 2.104 pfu of A/Scotland/20/74 (H3N2) virus (IAV) and treated or not 2 hpi with 3.4 mM of cis-aconitate. hIL-6 was measured in PCLS supernatant at 48 hpi (FIG. 4l). Data are represented as the mean±SEM. Statistical analysis was performed using the Kruskal-Wallis with Dunn's multiple comparison test.

FIG. 5. Cis-aconitate (CA) decreases viral load, lung inflammation and tissue damages in influenza virus-infected mice.

(FIG. 5a-5d) 7-week-old female C57Bl/6 mice were infected intranasally with 200 pfu of A/Scotland/20/74 (H3N2) virus (IAV) and treated or not 2 days post-infection with 0.6 mg of cis-aconitate (CA) intranasally. At 4 or 8 days p.i., mice were euthanized. At day 4 p.i., lungs as well as BALs fluids were collected to determine: (FIG. 5a) the viral load by a plaque forming unit assay (n=11); (FIG. 5b) the levels of 50 mediators using a specific protein-array on BAL fluids. At day 8 p.i., the number and activation of immune and inflammatory cells were determined by flow cytometry in BAL fluids (FIG. 5c) and lungs (FIG. 5d). All data are represented as the mean±SEM and are cumulative of 3 (FIG. 5a) or 2 (FIGS. 5c,5d) independent experiments.

(FIG. 5e-5h) 9 NF-kB transgenic Balb/c mice were infected with 300 PFU of A/Scotland/20/74 (H3N2) IAV and treated 2 days pi with 0.6 mg of CA intranasally. At 8 days pi, mice were anesthetized and luciferin was intra-nasally instilled (0.75 mg·kg⁻¹). (FIGS. 5g, 5h) Bioluminescence was measured using the IVIS imaging system. (FIG. 5e, f) Lung sections were stained with hematoxylin-eosin and tissues lesions were further assessed by microscopy and inflammation scoring. Scale bar: x6/20 μM. Statistical analysis was performed using the Mann-Whitney test.

FIG. 6. Cis-aconitate protects mice from influenza infection more efficiently than Oseltamivir and in late treatment.

7-week-old female C57Bl/6 mice were infected intranasally with 200 pfu of A/Scotland/20/74 (H3N2) virus (IAV) and treated (CA) or not (PBS) 20 minutes post-infection (FIG. 6a,6b) or 2 days post-infection (pi) (FIG. 6c,6d) with 30 mg/kg of cis-aconitate (CA) or 20 mg/kg of Oseltamivir (Osel) (by the intranasal route). Animal survival (FIG. 6a,6c) and body weight loss (FIG. 6b, 6d) were monitored daily. All data are represented as the mean±SEM and are cumulative of 1 (FIG. 6a, 6b) or 2 (FIG. 6c, 6d) independent experiments. Statistical analysis was performed using the Log-rank (Mantel-Cox) test.

FIG. 7. Cis-aconitate protects mice from influenza infection in a time frame consistent with patient care.

Patients hospitalized for community acquired pneumonia (CAP) were studied and presented in Table 1 and 2 (see page 27). Influenza refers to A and B virus strains. The time between the beginning of the onset of the symptom and the first hospital admission is presented in FIG. 7a. Each dot is a patient; the line represents the median. 7-week-old female C57Bl/6 mice were infected intranasally with 200 pfu of A/Scotland/20/74 (H3N2) virus (IAV) and treated (blue) or not (green) 4 and 5 days pi (FIG. 7b) with 30 mg/kg of cis-aconitate (CA) or 20 mg/kg of Oseltamivir (Osel) (by the intranasal route). Animal survival were monitored daily (FIG. 7b). Data are represented as the mean±SEM and are cumulative of 3 independent experiments. Statistical analysis was performed using the Log-rank (Mantel-Cox) test.

FIG. 8. Bronchial epithelial cells tolerance to cis-aconitate exposure.

(FIG. 8a) Human bronchial epithelial BEAS-2B cells were treated with PBS or with 3.4 mM of cis-aconitate (CA), itaconate (Ita), oxaloacetate (Oxa), isocitrate (IsoC), citrate (Cit), fumarate (Fum), pyruvate (Pyr) or glucose (Glc) for 16 h and cytotoxicity was assessed by MTS assay. BEAS-2B cells were treated (CA+) or not (CA-) with 3.4 mM of CA for 6 or 24 h (FIG. 8b, 8c, 8d) or with 0, 1.2, 2.3 or 3.4 mM of CA for 20 h (FIG. 8e). Cell proliferation (FIG. 8b), mitochondrial labeling (FIG. 8c), ROS production (FIG. 8d) and cell viability (FIG. 8e) were analyzed by Ki67, mitotracker, DHR 123 and Live Dead staining respectively. (FIG. 8f) Human primary bronchial epithelial cells (PBEC) were treated with 0.6, 3.4 or 5.7 mM CA for 16 h and cytotoxicity was assessed by a MTS test. Data are represented as mean±/−SEM of 4 (FIG. 8b-f) or 5 (FIG. 8a) independent experiments. Statistical analysis was performed using the Kruskal-Wallis test with Dunn's multiple comparison test (FIGS. 8a, 8e and 8f) or Friedman test with Dunn's multiple comparison test (FIG. 8b-8d).

FIG. 9. Tolerance and safety of cis-aconitate in vivo.

7-week-old female and males C57Bl/6 mice were instillated intranasally with PBS or 0.6 mg of cis-aconitate (CA) every second day for 15 days. (FIG. 9a) Body weight loss was monitored (n=15). Mice were euthanized at 15 days and lungs, bronchoalveolar lavage (BAL) fluids and serum were collected to determine: (FIG. 9b) the levels of 111 additional mediators in BAL using a specific protein-array; (FIG. 9c) the levels of ALAT activity in serum; (FIG. 9d) Microbiota alterations were assessed in mouse fecal pellets, after genomic DNA extraction and 16S rRNA sequencing. The diversity in microbial communities was quantified using Aitchison distance values, a measure of beta diversity. Each point represents the Aitchison distance between a CA-treated mouse and a PBS-treated mouse at day 0, 7, or 14. There are no differences in the overall fecal microbial composition between control and CA-treated mice over time (PERMANOVA; p<0.05). (FIG. 9e) the number and activation of immune cells in BAL by flow cytometry, and complete blood count (FIG. 9f). All data are represented as the mean±SEM and are cumulative (FIG. 9a,9c) or representative (FIG. 9b,9e) of 3 independent experiments. Statistical analysis was performed using Kruskal-Wallis test with Dunn's multiple comparison test.

FIG. 10. Cis-aconitate inhibits pro-inflammatory responses in SARS-CoV-2-infected epithelial cells.

(FIG. 10a) Epithelial cells were infected with severe acute respiratory syndrome coronavirus 2 (SARS-CoV-2) at MOI=1. Four hours post-infection, cells were washed and treated or not for 16 hours with 1 mg/ml of cis-aconitate. Levels of IL-6 were measured by ELISA in the cell supernatants. FIG. 10b: Vero epithelial cells were infected with Wuhan strain of SARS-CoV-2. FIG. 10c: Human primary bronchial epithelial cell were infected with Delta strain of SARS-CoV-2. Data are represented as the mean±SEM and are cumulative of 3 independent experiments. * for p<0.05, ns: non-significant.

FIG. 11. Effect of cis-aconitate on inflammation in vivo.

NF-kB transgenic Balb/c (FIG. 11a, 11b) or C57Bl/6 (FIG. 11c) mice were instilled with 10 μg of lipopolysaccharides (LPS) and treated 15 minutes post-stimulation with 0.6 mg of cis-aconitate (CA) intranasally. 24 h post-instillation, mice were anesthetized. In the group of NF-kB transgenic Balb/c animals, luciferine was intranasally instilled (0.75 mg·kg⁻¹) and bioluminescence was measured using the IVIS imaging system (FIG. 11a, 11b). In the group of C57Bl/6 mice, BALs fluids were collected to determine TNFα levels (FIG. 11c).). Bioluminescence was then measured using the IVIS system. All data are represented as the mean±SEM and are 1 independent experiments. Statistical analysis was performed using the Mann-Whitney test.

FIG. 12. Confirmation of the protective effects of cis-aconitate in IAV-infected Balb/c mice.

To validate the findings observed in C57Bl/6 mice (refer to FIGS. 6 a-d) within a different mouse species, we conducted an experiment on Balb/c mice. These (NF-kB transgenic) Balb/c animals were infected with 300 PFU of A/Scotland/20/74 (H3N2) IAV and treated 2 days pi with 0.6 mg of cis-aconitate (CA) intranasally. At 8 days pi, mice were anesthetized and luciferine was intra-nasally instilled (0.75 mg·kg⁻¹). Animal survival (FIG. 12a) and body weight loss (FIG. 12b) were monitored daily. All data are represented as the mean±SEM. Statistical analysis was performed using the Log-rank (Mantel-Cox) test.

FIG. 13. Anti-influenza activity of cis-aconitate is independent from itaconate. CAD-deficient and WT C57Bl/6 mice were infected intranasally with 100 pfu of A/Scotland/20/74 (H3N2) virus (IAV) and treated intranasally or not 20 minutes post-infection with 30 mg/kg of cis-aconitate (CA) Animal survival was monitored daily. All data are represented as the mean±SEM. Statistical analysis was performed using the Log-rank (Mantel-Cox) test.

### EXAMPLES

### Example 1: Mitochondria-Derived Metabolite Cis-Aconitate Protects Against Influenza Mortality Through Dual Antiviral and Anti-Inflammatory Activities

### 1.1/Material and Methods

Viruses. Mouse adapted-influenza A/Scotland/20/74 (H3N2) was generously given by Pr. Sylvie van der Werf's team (Pasteur Institute, Paris, France). The influenza A/PR/8/34 (H1N1) strains were kindly provided by Dr. Georg Kochs (Freiburg University, Germany). H1N1 pandemic IAV strain were generously given by Dr. François Trottein (Center for Infection and Immunity of Lille). The influenza B/Paris/234/2013 (Yamagata) lineage was acquired from the European Virus Archive Global (EVAg).

Cell culture. In vitro experiments were performed using human bronchial epithelial BEAS-2B cells, except for plaque assays which used Madin-Darby Canine Kidney (MDCK) cells, and minigenome assay which used HEK-293T. These cells are cultured in either F-12K Medium (BEAS-2B) or MEM (HEK-293T and MDCK) supplemented with 10% FBS and 100 U/mL penicillin, 100 μg/mL streptomycin. All cells were mycoplasma-free. BEAS-2B cells were infected in medium without FBS for 4 hours with IAV Scotland at MOI=1 (except for TEM and SEM analysis, for which an MOI=5 was applied). Cells were also stimulated in medium with FBS with 2 μg/mL of Poly (I:C) or in medium without FBS with 2 μg/mL of PMA or 20 ng/ml of TNFα. Four hours after the challenge, cells were washed with PBS and subsequently exposed to varying concentrations of cis-aconitate or other tested metabolites for either 4 or 16 hours.

Neuraminidase (NA) assay. The assay measures the release of a 4-methylumbelliferone fluorescent product from the 2′-(4-Methylumbelliferyl)-α-D-N-acetylneuraminic acid sodium salt hydrate (MU-NANA) substrate. 67 μL of cell supernatant was incubated with 33 μL of MU-NANA (50 μM) in black 96-well micro-plates. Fluorescence was immediately measured in a kinetic assay over 1 h at Ex=355 nm and Em=460 nm.

Protein-array and ELISA. Cells supernatants or BAL were centrifuged 5 min at 500 g and supernatants were stored at −80° C. Protein array and DuoSet ELISA (Human IL-6, and mouse MPO and ALT) were performed according to the manufacturer's (R&D Systems or Clinisciences for ALT ELISA) instructions.

Minigenome assay. The minigenome study was performed in 24-well plates. Briefly, 293T cells were transfected with together with 50 ng of pRF483-PA-RT, 50 ng of pRF483-PB2-RT, 100 ng of pRF483-NP-RT, 50 ng of pRF483-PB1-RT and 150 ng of reporter plasmid pPoll-WSN-NA-firefly luciferase which contains a firefly luciferase ORF flanked by the noncoding regions of the NA segment under the control of human polymerase I promoter. As a negative control, 293T cells were transfected with the same plasmids, with the exception of the PB1 expression plasmid. The procedure used the Fugene HD transfection reagent (Promega) according to the manufacturer's instructions. 20 h post-transfection, cells were treated with different concentrations of cis-aconitate. 48 post-transfection, cells were washed twice with phosphate-buffered saline (PBS) and lysed in 100 μl of lysis buffer provided with the Firefly Luciferase Assay System (Promega). Firefly luciferase activities were measured on 20 μl of cell extracts, using the Firefly luciferase substrate provided with the above-mentioned kit and a Centro luminometer (Berthold).

RNA isolation and RT-qPCR. Cells in 6-well plates were lysed with 350 μL of “RA1” buffer (included in the Macherey-Nagel RNA extraction kit) and β-mercaptoethanol diluted 1/100. Total RNAs from cells were extracted using the NucleoSpin® RNA kit, including a step of genomic DNa digestion with DNase. Nucleic samples were quantified using a Nanodrop 2000 UV-visible spectrophotometer. For each sample, single-stranded cDNA was synthesized from 500 ng total RNA with the High Capacity cDNA reverse transcription kit (Applied Biosystems), using the specific sense IAV M1 primer or random primers. mRNA levels were determined using quantitative real-time PCR with a LightCycler 480 instrument (Roche Diagnostics). PCR was carried out using 10 ng of reverse-transcribed total RNA as the template, 10 μM (each) forward and reverse primers, and 10 μL SYBR® Premix Ex Taq in a final volume of 20 μL. Each reaction was performed in duplicate in white 96-well plates. The thermal protocol consisted of an initial denaturation step at 95° C. for 30 s followed by 40 cycles of denaturation at 95° C. for 5 s and primer annealing and extension at 60° C. for 20 s (reading at 83° C.). For each amplified cDNA, melting curves were generated to check the reaction specificity.

Western-blotting. Cells in 6-well plates were lysed with 150 μL of RIPA buffer (150 mM sodium chloride, 50 mM Tris-HCl, 1 mM ethylenediaminetetraacetic acid, 1% Triton X100, 1% sodium deoxycholic acid, 0.1% sodium dodecyl sulphate) and a protease inhibitor cocktail (diluted 1/200) or PhosphoSafe Extraction Reagent. Samples were centrifuged for 10 min at 12,000 g to eliminate debris. Protein concentrations were measured using a Pierce™ Protein BCA Assay Kit. Ten μg of total proteins were diluted with reducing Laemmli buffer, heated at 100° C. for 5 min, and loaded onto 12% SDS-PAGE. Proteins were subsequently transferred to nitrocellulose membranes, and probed with anti-NP (1/500), anti-NS1 (1/1000), anti-PA (1/1000), anti-PB2 (1/500), anti-M1 (1/1000), anti-(P) ERK1/2 (1/2000), anti-(P) AKT (1/1000), anti-(P) p65 (3000) or anti-β-actin (1/5000). Bound antibodies were revealed with an anti-rabbit IgG for NS1, PB2, (P) AKT, (P) ERK1/2 and (P) p65 and anti-mouse IgG for other proteins (HRP linked) and ECL detection reagents. An automated imaging system (MF ChemiBis 3.2, DNR BioImaging Systems) was used for detection, and the FUJI FILM MultiGauge software was subsequently used for analysis and quantification.

Confocal fluorescence microscopy. Cells were grown in 12-well plates with a cover slide in the bottom of the well. After different treatments, cells were fixed using 4% formaldehyde for 30 min at room temperature and then permeabilized in PBS 0.1% Triton X-100 for 30 min at room temperature. Following a 1 h saturation step with PBS 1% bovine serum albumin, 0.1% Tween 20, cells were stained for 2 h at room temperature with anti-NP-FITC (1/30), anti-NS1 (1/200), anti M1 (1/150), anti-PB2 (1/200) and anti-PA (1/50) antibody. An anti-rabbit-AF488 was used for 2 h at room temperature as the secondary antibody for NS1 and PB2, and an anti-mouse-AF488 was used for other proteins. Then actin was stained with ActinRed 555 reagent for 30 min and nuclei were stained with the NucBlue reagent for 5 min. Samples were analyzed with a Leica SP8 confocal microscope and Leica LasX Life Sciences Software.

Transmission Electron Microscopy. Cells were washed with PBS, detached using trypsin and centrifuged. Cells were fixed by incubation for 24 h in 4% paraformaldehyde, 1% glutaraldehyde in 0.1 M phosphate buffer (pH 7.2). Samples were further washed in PBS and post-fixed by incubation with 2% osmium tetroxide for 1 h. Next, samples were fully dehydrated in a graded series of ethanol solutions and propylene oxide. An impregnation step was performed with a mixture of (1:1) propylene oxide/Epon resin and then samples were left overnight in pure resin. They were then embedded in Epon resin, which was allowed to polymerize for 48 h at 60° C. Ultra-thin sections (90 nm) of these blocks were obtained using a Leica EM UC7 ultramicrotome (Wetzlar, Germany). Sections were stained with 2% uranyl acetate and 5% lead citrate. Observations were made with a transmission electron microscope (JEOL 1011) and analyzed with a Digital Micrograph.

Scanning Electron Microscopy. Cells were washed with PBS, detached using trypsin and centrifuged. Cells were fixed by incubation for 24 h in 4% paraformaldehyde, 1% glutaraldehyde in 0.1 M phosphate buffer (pH 7.2). Samples were then washed in phosphate-buffered saline (PBS) and post-fixed by incubation with 2% osmium tetroxide for 1 h. Samples were then fully dehydrated in a graded series of ethanol solutions and dried in hexamethyldisilazane (HMDS, Sigma, St-Louis, MO). Finally, the dry sample was sprinkled onto carbon disks and coated with 40 Å platinum, with a GATAN PECS 682 apparatus (Pleasanton, CA), before observation under a Zeiss Ultra plus FEG-SEM scanning electron microscope (Oberkochen, Germany).

IAV titration by plaque-forming units assay. Titrations in cell supernatants and mouse lungs were performed as previously described [38].

Human primary bronchial epithelial cells culture. Human primary bronchial epithelial cells (PBEC) were isolated from macroscopically normal bronchial tissues obtained from patients undergoing lobectomy at the university hospital (“CHRU”) of Tours (Tours, France). Cancer-free trimmed tissues were washed and incubated 2 h at 37° C. with 0.018% (w/v) proteinase type XIV (Sigma-Aldrich) in Ca²⁺/Mg²⁺-free Hank's Balanced Salt Solution (Gibco). Epithelial cells were gently scraped off the luminal surface, washed and subsequently cultured in serum-free keratinocyte medium (Gibco) supplemented with 2.4 ng/ml epidermal growth factor (Gibco), 25 μg/ml bovine pituitary extract (Gibco), 1 μM isoproterenol (Sigma-Aldrich), 100 U/mL Penicillin and 100 mg/ml Streptomycin (Lonza) on coated 6-well plates (coated at 37° C., 5% CO2 for 24 h with 30 μg/ml PureCol (Advanced BioMatrix, San Diego, CA, USA), 10 μg/ml Bovine serum albumin (Sigma-Aldrich) and 5 μg/ml fibronectin (isolated from human plasma and diluted in PBS). During the first week of culture, 1/500 Primocin (Invivogen, France) was added to the medium. After reaching near-confluence, cells were trypsinized (0.03% [w/V] soft trypsin (Gifco, Detroit, USA), 0.01% (w/v) EDTA (VWR, France), 0.1% glucose (VWR, France) in PBS) and stored in liquid nitrogen. These PBEC were used for generation of mucociliary differentiated cells by differentiation in liquid culture as described previously [83], excepted that instead of using BEGM/DMEM medium, we used PneumaCult EX of Stemcell. Prior to the stimulation of PBEC with either 2 μg/ml of PIC or 50 mM of PMA, a preliminary proliferation phase of 3-4 days was conducted using PneumaCult EX medium. The subsequent stimulations were performed in a medium composed of a 1:1 mixture of BEGM medium and complete DMEM/F12 medium. This medium was supplemented with 100 U/mL penicillin, 100 μg/mL streptomycin, 12.5 ml of Gibco™ HEPES (1M), and 5 ml of Gibco™ GlutaMAX™ Supplement

Human precision-cut lung slices (PCLS). PCLS were performed on lung resections from surgical patients of CHRU of Tours. Lung explants were sliced using Mcllwain Tissus choper into 400 μm thin slices. Individualized PCLS were placed in air-liquid interface and infected with 2.104 pfu of A/Scotland/20/74 (H3N2) virus (IAV). After 2 hours, PCLS were treated with 3.4 mM of cis-aconitate and maintained at 37° C., 5% CO2. At 48 h post-infection, the PCLS supernatants were harvested to measure cytokine release. PCLS were fixed in formalin for tissue imaging.

Animal infection and fluid collection. 7-week-old female or male C57Bl/6 mice were infected intranasally or not with 200 pfu of A/Scotland/20/74 (H3N2) IAV, and treated or not with 0.6 mg of cis-aconitate at different time points. On the day of sacrifice, blood was either collected and subjected to centrifugation for 10 minutes at 10,000 g to analyze the serum, or it was heparinized and examined using the ProCyte Dx hematocytometer (Idexx, France). Airways were washed four times with 0.5 ml of PBS for BAL collection. After centrifugation, BAL fluids were stored at −80° C. for subsequent measurement of inflammatory mediators and pellets were recovered in PBS 2% FBS. Erythrocytes were discarded using a red blood cell lysis buffer and leukocytes were counted and analyzed by flow cytometry. After BAL, lungs were perfused with 10 mL PBS injected into the heart. Left lung was placed in 4% paraformaldehyde in PBS for histological analysis. Lung suspensions were obtained by enzymatic digestion using gentleMACS dissociators (Miltenyi Biotech) according to the kit manufacturer's instructions. Groups of 9 BalB/c NF-kB transgenic immunocompetent mice were also infected with 300 PFU of the A/Scotland/20/74 (H3N2) IAV. At 8 days post-infection, mice were anesthetized and luciferine was intra-nasally instilled (0.75 mg·kg⁻¹) and luciferase activity was quantified. Bioluminescence was measured using the IVIS imaging system. Body weight loss was monitored daily and mice are sacrificed when they reached a weight loss of 25% of their initial weight.

Groups of 20 wild-type (WT) or cis-aconitate decarboxylase (CAD)-deficient C57Bl/6 mice were infected with 100 PFU of the A/Scotland/20/74 (H3N2) IAV and treated or not with 0.6 mg of cis-aconitate 20 min pi. Body weight loss was monitored daily and mice are sacrificed when they reach a weight loss of 20% of their initial weight.

Flow cytometry analysis. BAL or human bronchial epithelial cells were dispensed into round bottomed 96-well plates and were centrifuged at 300 g at 4° C. for 5 min. Samples were further stained using specific antibodies and appropriate isotype controls. For each antibody, one well was seeded for the Fluorescence Minus One Control. Flow cytometry data were acquired on a MACSQuant® Analyzer and analyses were performed using the VenturiOne software.

Histopathology. Lungs were collected after BAL and airways were washed, and placed in 4% paraformaldehyde in PBS. Lung sections of approximately 4 μm thickness were cut and stained with hematoxylin-eosin. A study pathologist examined the tissue sections using light microscopy on a Leica Diaplan microscope in a blinded experimental protocol. All histopathological findings were graded in a semi-quantitative fashion on a scale of 0 to 4 (0: absent, 1: mild, 2: moderate, 3: severe, 4: extremely severe). All lung preparations and analyses were performed at the LAPV (Amboise, France).

Genomic DNA Extraction. Genomic DNA was extracted from mouse fecal pellets by transferring the pellet into a tube containing 2.8 mm ceramic beads (VWR), 0.1 mm glass beads (VWR), 100 μl of GES, and 800 μl of 200 mM sodium phosphate buffer (pH 8). Samples were bead beat using the Powerlyzer 24 Bench Top Homogenizer for three minutes at 3000 revolutions per minute. After centrifugation for ten minutes at 15,000 rpm, the supernatant was processed using the MagMAX Express 96-Deep Well Magnetic Particle Processor (Applied Biosystems) with the DNA Multi-Sample kit (Life Technologies).

16S rRNA Sequencing and Analysis. PCR was used to amplify the v34 region of the 16S rRNA gene. The reaction consisted of 50 ng of template DNA, 5 pmoles of 341F and 806R Illumina adapted primers, 1 U of Taq polymerase, 1× buffer, 1.5 mM MgCl2, 0.4 mg/mL bovine serum albumin, and 0.2 mM each dNTPs. The PCR program included an initial denaturation at 94° C. for five minutes, then five cycles of 94° C. for 30 seconds, 47° C. for 30 seconds, and 72° C. for 40 seconds, followed by 25 cycles of 94° C. for 30 seconds, 50° C. for 30 seconds, and 72° C. for 40 seconds, and a final extension of 72° C. for ten minutes. Amplicons were normalized using the SequalPrep normalization kit (ThermoFisher) prior to sequencing on the Illumina MiSeq platform at McMaster's Genomics Facility. Cutadapt (DOI: 10.14806/ej.17.1.200) was used to trim raw reads based on a minimum quality score of 30, remove adaptors, and remove reads less than 100 bp. The DADA2 pipeline (10.1038/nmeth.3869) was used to determine amplicon sequence variants (ASVs) for individual Illumina runs. Sequence variant tables from each Illumina run were then merged and DADA2 was used to remove bimeras. The taxonomy of each ASV was assigned based on the SILVA database v1.3.2. Statistical analyses and visualization of the fecal microbiome data was performed using RStudio v4.1.2. ASVs unclassified at the kingdom or phylum level or ASVs classified as Eukaryota or Mitochondria were Aitchison distances were measured using the microbiome excluded. (http://microbiome.github.io) and phyloseq packages. (10.1371/journal.pone.0061217). Permutational multivariate analysis of variance (PERMANOVA) was performed using the adonis function in the vegan package (10.1111/j.1654-1103.2003.tb02228.x).

Cell proliferation and cytotoxicity assays. Cells in 96-well plates were washed twice with PBS and incubated for 1 h at 37° C. with 100 μL of MTS reagent diluted 1/5 for the cell proliferation test. Optical density was measured at 490 nm. For the cytotoxicity assay, cells were stained for 15 min at 4° C. with Live/Dead or with anti-Ki67, mitotracker and before flow cytometry analysis.

siRNA transfection. Beas-2B cells (1.25×10⁵) were seeded in a 12-well plate one day prior to siRNA transfection. The transfection utilized ON-TARGETPlus human smartpool IRG1 (Dharmacon) or MISSION® negative control scramble (Sigma-Aldrich) siRNA. Each siRNA stock was diluted to 50 nM in 100 μL of OptiMEM (Gibco) containing 1.5 μL of RNAiMax reagent (Invitrogen). After a 5-minute incubation at room temperature, 100 μL of each siRNA mix was added to 900 μL of fresh medium per well. RNA interference (RNAi) was conducted for 48 hours (with medium replacement after 24 hours), and gene knockdown was assessed by RT-qPCR.

Patient data collection. The study was performed in a 37-bed ICU of the University Hospital (“CHRU”) of Tours (France) over an 18-month period. Pneumonia was defined as the presence of an infiltrate on a chest radiograph and one or more of the following symptoms: dyspnea, or cough with or without sputum production, or fever (temperature ≥38.0° C.) or hypothermia (temperature <35.0° C.). Community-acquired infection was defined as infection occurring within 48 h of admission, excluding those with nosocomial pneumonia. Cases of pneumonia due to pneumocystis or inhalation were not included. Cases with PaO2≥60 mmHg in ambient air or with the need for oxygen therapy≤4 L/min or without mechanical ventilation (invasive or non-invasive) were not included. Baseline patient information was collected at case presentation through in-person semi-structured interviews with patients or surrogates. Observations from the physical examination at presentation, including vital signs and auscultation of the lungs, were recorded. Microbiological investigations included blood cultures, legionella and pneumococcal urinary antigen tests, bacterial cultures of tracheal aspirates, multiplex PCR RespiFinder SMART 22® (PathoFinder B.V., Oxfordlaan, Netherlands) analyses on respiratory fluids (sputum and/or nasal wash and/or endotracheal aspirate and/or bronchoalveolar lavage [BAL]).

Statistical analysis. Statistical analyses were performed using GraphPad Prism. Data are reported as mean±SEM. Statistical values, including the number of replicates (n) and the statistical test used, can be found in the figure legends. *p<0.05, **p<0.005, ***p<0.0005, ****p<0.0001. For in vitro experiments, n=the number of separate experiments. For in vivo work, n=the number of individual animals.

Study approvals. All animal experimentations were performed according to the national governmental guidelines and were approved by local and national ethics committee (CEEA. 19, #201604071220401-4885). Informed consent was obtained from each patient in compliance with the Helsinki declaration and the tissue and cell collections were declared to the French Ministry of Graduate study, Research and Innovation (DC-2008-308, MERI Ministère de l'Enseignement Supérieur, de la Recherche et de l'Innovation). Human data collection complied with French law for observational studies, was approved by the ethics committee of the French Intensive Care Society (CE SRLF 13-28), was approved by the Commission Nationale de l′Informatique et des Libertés (CNIL) for the treatment of personal health data. We gave written and oral information to patients or next-of-kin. Patients or next-of-kin gave verbal informed consent, as approved by the ethic committee.

### 1.2/Results

### Cis-Aconitate Inhibits Both Influenza A Virus Replication and Production of Inflammatory Mediators in Infected Lung Epithelial Cells.

To detect potential anti-influenza effects among metabolites derived from glycolysis or TCA cycle, we infected human bronchial epithelial cells (BEAS-2B cells) with influenza A virus (IAV) (A/Scotland/20/74, H3N2) and treated 4 hours post-infection (hpi) with 3.4 mM of metabolite. This concentration corresponds to the highest dose for which no cytotoxic effects were observed for all tested metabolites (FIG. 8a). As a surrogate of the release of neo-virions, we measured the neuraminidase (NA) activity in cell supernatants at 20 hpi. We observed a 60-fold reduction (p=0.0003) of NA activity in infected cells treated by cis-aconitate (FIG. 1b). A reduction of NA activity was also observed with itaconate (4.8-fold, p=0.028). Other tested metabolites did not have any effect on NA activity.

Consistently, cis-aconitate reduced interleukin 6 (IL-6) production (an inflammatory mediator) of IAV-infected epithelial cells (FIG. 1c).

Cis-aconitate is a TCA intermediate that is critical to produce energy in the form of ATP. In addition, cis-aconitate decarboxylase (CAD, also known as ACOD1 or Irg1) converts cis-aconitate to itaconate. To ensure that the cis-aconitate anti-influenza effect was not attributable to itaconate, we used CAD siRNA to prevent the cis-aconitate metabolization to itaconate by BEAS-2B cells. CAD siRNA efficiently block CAD expression compared to control scramble siRNA (FIG. 1d), but cis-aconitate antiviral and anti-inflammatory properties are conserved (FIG. 1e-g). This underscores that cis-aconitate protects epithelial cells from IAV infection regardless of itaconate involvement, revealing its standalone protective mechanism.

Supplementary tests also showed that trans-aconitic acid exhibited similar antiviral and anti-inflammatory activities, indicating that both isomers of aconitic acid possess anti-influenza properties (FIG. 1h-i).

### Cis-Aconitate Inhibits the Influenza Polymerase.

To better understand how cis-aconitate inhibits the production of viral particles, we studied its impact on different stages of IAV replication cycle. The IAV is an enveloped virus with a genome made up of negative sense, single-stranded, segmented RNA. It has eight segments that encode for the 12 viral genes. The influenza virus life cycle can be divided into the following stages: (i) entry into the host cell; (ii) entry of viral ribonucleoproteins (vRNP) into the nucleus; (iii) transcription and replication of the viral genome; (iv) translation of messenger viral RNA into viral proteins; (v) export of the vRNP from the nucleus; and (vi) assembly and budding at the host cell plasma membrane. We explored backward all these steps.

Thus, we first examined IAV-infected cells by transmission and scanning electronic microscopy (TEM, SEM, respectively) and observed a marked reduction of virus budding upon cis-aconitate treatment (IAV+CA, FIG. 2a). Whether the reduction of virus budding was explained by an impaired budding process per se or was the consequences of a decrease production of viral material (viral RNA and/or proteins) was unknown. We quantified both the expression of viral proteins and mRNA in infected epithelial cells treated or not with cis-aconitate. Using western-blot and confocal microscopy analysis, we observed that viral protein expression of NP, NS1, and PA were drastically decreases at 8 hpi in treated condition (FIG. 2b-e). Using qRT-PCR, we showed that relative expression of viral matrix 1 (M1) mRNA was decreased by tenfold by cis-aconitate treatment at 6 h pi (p<0.01, FIG. 2f). Delivering the vRNPs from the cell surface to the nucleus lasts in approximately 1 h, with entry and fusion occurring rather quickly (~10 min) [33, 34]. Since the cis-aconitate was administered 4 hpi after the infection (and after cell wash steps), we assumed that it was unlikely that cis-aconitate impacts the early steps of viral entry, from cell fusion to nuclear trafficking. Overall, these results suggested that the transcription of the viral genome may be inhibited by the cis-aconitate and that the decrease in viral protein expression and ultimately neo-virions production were down-stream effects. To test the effect of cis-aconitate on viral polymerase activity we conducted a minigenome assay on 293T cells that were transfected with reporter plasmid pPoll-WSN-NA-firefly luciferase. After 24 hours of treatment, we observed a dose-dependent decrease in luciferase activity, which was reduced by ~75% with 3.4 mM of cis-aconitate (p<0.03, FIG. 2g). Overall, we demonstrated that cis-aconitate disrupts the influenza virus life cycle by inhibiting viral polymerase activity.

### Cis-Aconitate Presents Antiviral Properties Against Various Types and Subtypes of Influenza Viruses

All previous data were generated with IAV (A/Scotland/20/74 (H3N2)) infection. However, influenza viruses show great diversity and a large number of subtypes. In 2018, the worldwide center for surveillance, epidemiology, and control of influenza reported that influenza A was the main type, with A (H1N1) pdm09 (corresponding to the 2009 H1N1 pandemic strain) and A (H3N2) predominating [35]. Influenza B viruses also represent an important part of influenza infections. Thus, 35% of respiratory specimens tested positive for influenza in 2013 were positive for influenza B viruses, with 80% belonged to the B/Yamagata lineage and 20% to the B/Victoria lineage. Consequently and in addition to influenza strain A/Scotland/20/74 H3N2 tested previously, we evaluated the impact of cis-aconitate on influenza strain A/pandemic/2009 H1N1 (FIG. 3a), influenza strain A/Puerto Rico/8/1934 H1N1 (FIG. 3.b), and influenza B Yamagata strain B/Paris/234/2013 (FIG. 3.c). Plaque-Forming Units assays show at least one log decrease of viral particles production by cis-aconitate, whatever the types or subtypes of influenza viruses used (FIG. 3a-c). These data suggest broad spectrum antiviral properties of cis-aconitate among influenza viruses.

### Cis-Aconitate is an Anti-Inflammatory Mediator.

We wondered if the decrease of inflammatory mediator production (IL-6) by cis-aconitate was a downstream effect of its antiviral effect or was the consequence of inherent immunomodulatory properties. This question is of importance regarding the influenza physiopathology which is both the result of cytopathic viral effects and excessive inflammatory responses (when the balance of immunity is shifted to excess then immunopathology occurs) [36-38].

Harmful immune response can be characterized by dysregulated activations of intracellular signalling pathways and overproduction of inflammatory cytokines [39-41]. In IAV infected-cells, phosphorylation of Ras-dependent extracellular signal-regulated kinase (ERK)1/2, Protein kinase B (PKB), also known as Akt, and the p65 subunits of the nuclear factor-kappa B (NF-κB) were up-regulated compared to non-infected condition at 20 hours pi (FIG. 4a). The accumulation of these phosphorylated forms was inhibited by cis-aconitate treatment in infected-cells. Consistently, IAV infection induces 4- to 7-fold increase expression of CCL2/MCP-1, CCL5/RANTES, CXCL1/GROa, CXCL10/IP-10, IL-6 and CXCL8/IL-8 compared to controls (FIG. 4b, green bars) that were inhibited by cis-aconitate (FIG. 4b, blue bars).

Interestingly, the treatment of non-infected lung epithelial cells with cis-aconitate resulted also in reductions of critical signaling pathways (FIG. 4a, inhibition of ERK, AKT and NF-κB signaling pathways by cis-aconitate). Of note, the doses of cis-aconitate used did not impact cell proliferation, mitochondria accumulation, ROS production or cell viability (FIG. 8b-8e). To gain insight into the cis-aconitate intrinsic immunoregulatory properties, we stimulated bronchial epithelial cells with different inflammatory agonists: (i) Poly (I:C) (PIC), a synthetic double-stranded RNA poly(I:C) and a potent agonist of TLR3 signaling that mimics IAV-triggered immune responses [42]; (ii) Phorbol 12-myristate 13-acetate (PMA), a diester of phorbol that activates the signal transduction enzyme protein kinase C (PKC); (iii) TNFα, an inflammatory cytokine responsible for a diverse range of signaling events within cells, leading to necrosis or apoptosis. All these agonists induced activation of signaling pathways leading to the release of inflammatory cytokines. We observed that cis-aconitate treatment induces trend (FIG. 4c, TNFα) or significant decrease (FIG. 4c, IAV, PIC, PMA) of IL-6 production in dose-dependent manner.

Collectively, these findings demonstrate that cis-aconitate exhibits anti-inflammatory properties that can effectively disrupt the inflammatory cascades involved in influenza infection.

Anti-Infective and Anti-Inflammatory Properties of Cis-Aconitate were Confirmed in Preclinical Models

To gain insight into the generalization and translation of the previous observations, we explored whether the antiviral and anti-inflammatory properties of cis-aconitate were confirmed in experimental models that have complementary approaches: ex vivo models of infection in human bronchial epithelial cells and human lung slices as well as in in vivo murine model of influenza pneumonia.

### Effect of Cis-Aconitate in Human PEBCs and Lung Tissues.

Human primary bronchial epithelial cells (PBEC) were isolated from bronchial tissues obtained from patients undergoing lobectomy at the CHRU of Tours Center and were cultured reproducing ex vivo a pseudo-stratified airway epithelium with basal cells, mucus producing cells and ciliated cells [43, 44]. Consistently, this model is highly relevant for studying viral infections [45-48]. We observed that, after influenza infection, cis-aconitate treatment significantly decreased NA activity (FIG. 4e). IAV induced a substantial hIL-6 production (~1300 μg/mL) which was markedly and dose-dependently inhibited by cis-aconitate (up to 80% reduction, FIG. 4f). Additionally, the expression of pro-inflammatory cytokines induced by the immunostimulants PIC or PMA showed a dose-dependent decrease with cis-aconitate treatment (FIG. 4g, 4h). Given the pivotal role of cell death in Influenza A Virus (IAV) pathogenesis ([62]), we meticulously assessed the impact of cis-aconitate on this critical cellular process using state-of-the-art Incucyte® technology. Notably, the application of cis-aconitate resulted in a robust reduction (approximately 70%) in SYTOX™ labeling, a marker indicative of dead cells, unveiling that cis-aconitate has a significant protective effect against IAV-triggered cell death (FIG. 4i, 4j). Finally, we examined the anti-inflammatory effects of cis-aconitate on freshly collected human lung tissue infected ex vivo with IAV. As expected, IAV infection led to the secretion of pro-inflammatory cytokines in human precision-cut lung slices (PCLS; FIG. 4k). Notably, treatment with cis-aconitate resulted in a significant twofold reduction in IL-6 production (FIG. 4l). These findings collectively affirm the dual antiviral and anti-inflammatory attributes of cis-aconitate within ex vivo human models, underscoring its high translational relevance.

### Anti-Inflammatory Effect of Cis-Aconitate In Vivo

Then, we further examined the anti-inflammatory effect of cis-aconitate administered intranasally in a valuable murine model of acute lung injury induced by lipopolysaccharide ([63]), a cell wall component characteristic of Gram-negative bacteria.

However, prior to these experiments, the safety profile of cis-aconitate was thoroughly assessed through chronic instillation experiments in mice, spanning a 15-day period. Notably, no adverse effects or alterations in microbial composition were observed under these conditions (FIG. 9a-9d). Subsequently, we evaluated the activity of NF-kB, a key transcription factor in inflammatory signaling, induced by LPS instillation in NF-kB-luciferase transgenic mice, with or without cis-aconitate treatment (FIG. 6a-b). At 24 hours post-stimulation, the inflammation induced by LPS (Figure. 11a, center picture) was markedly reduced in cis-aconitate-treated animals (FIG. 11a, right picture, and FIG. 11b). Consistent with this observation, TNFα secretion in the BAL fluids of mice challenged with LPS significantly decreased in those treated with cis-aconitate (FIG. 11c). Thus, our in vivo experiments confirmed the inhibitory effect of cis-aconitate on inflammatory pathways, leading to a reduction in downstream pro-inflammatory cytokine secretion.

### Therapeutic Effect of Cis-Aconitate in Influenza Virus-Infected Mice

Next, we examined the therapeutic potential of cis-aconitate in a murine model of acute IAV pneumonia. Mice were infected intranasally with a lethal dose of influenza A/Scotland/20/74 (H3N2) and treated with 0.6 mg of cis-aconitate two days post-infection. In the initial phase, mice were sacrificed at four days post-infection to analyze early events in influenza physiopathology. Notably, cis-aconitate-treated mice exhibited a significant reduction in viral load in lung tissues compared to control animals (1-log reduction, p<0.0001; FIG. 5a). Further assessment involved the examination of the relative expression of 50 mediators in the bronchoalveolar lavage (BAL) compartment, encompassing pro-inflammatory cytokines, interferons, stimulating or growth factors, and proteases; FIG. 5b). In response to IAV infection, all these mediators increased, yet were markedly reduced in cis-aconitate-treated animals (FIG. 5b).

Extending the sacrifice time to eight days post-infection, our model exhibited characteristics of severe IAV-induced lung injury [49-53], including in BAL fluids an increased number of leukocytes, an alveolar macrophage depletion, a neutrophil recruitment, and activation of T lymphocytes, NKT cells, and DC (FIG. 5c (BAL fluids), 5d (lung tissues)). Cis-aconitate treatment consistently mitigated these responses: alveolar macrophage numbers were comparable to non-infected conditions, while neutrophil recruitment decreased by two-thirds (p<0.02, FIG. 5d) and activation of NKT, DC, alveolar macrophages, and T cells was significantly lower compared to what was observed in infected, non-treated mice (FIGS. 5c and 7d).

The observed trend with cis-aconitate in normalizing cellular infiltration and immune cell activation is likely to have beneficial effects on lung tissue, considering the damage these events can cause in severe IAV infection [54-59]. Consistently, histopathological analysis revealed a significant reduction in IAV-induced alveolar wall thickening, hyaline membrane formation, and epithelial necrosis in mice treated with cis-aconitate compared to untreated animals (FIG. 5e,f). Treated mice demonstrated diminished lung congestion and a reduced inflammation score compared to untreated mice (FIG. 5e right panel, and 5f).

To further evaluate IAV-triggered inflammation on a broader scale, we measured NF-kB activity in NF-kB-luciferase transgenic mice infected with IAV and treated with or without cis-aconitate (FIGS. 5g and h). At 8 days post-infections, the inflammation induced by influenza infection spreads systemically (i.e. inflammation of the digestive tract; FIG. 5g, center picture). This inflammation was drastically reduced in cis-aconitate treated mice (FIG. 5g, right picture, and h).

Thus, by reducing all components of influenza pathogenesis, including viral replication, secretion of inflammatory mediators and recruitment/activation of inflammatory cells, cis-aconitate effectively prevents lung damages induced by IAV.

### Cis-Aconitate Mitigates Mortality in IAV-Infected Mice Across an Extended Treatment Window

To determine the anti-influenza drug potential of cis-aconitate, we evaluate its benefit on mortality in infected mice model, in comparison with the anti-influenza drug reference, oseltamivir (neuraminidase inhibitor) [60]. IAV-infected but untreated mice displayed a zero survival rate, while mice treated with cis-aconitate or oseltamivir at 20 minutes post-infection reached 80% and 90% survival rates, respectively (FIG. 6a). After 15 days, the surviving mice had returned to their original weight. Delaying oseltamivir administration to 2 days post-infection nullified the treatment's beneficial effect on the survival of infected mice (FIG. 6c). Remarkably, the protective effect of cis-aconitate persisted even when treatment was postponed to 2 days post-infection in both C57Bl/6 and Balb/c mice (FIG. 6c,d).

Exploratory research should ultimately improve patient care. For this to be possible, experimental designs must consider settings that genuinely reflect real-world concerns. The time to appropriate anti-infective therapy is critical as it is a predictor of outcome. During influenza infection, there is a delay between the progressive onset of the symptoms and the actual consultation or hospitalization for treatment administration. Thus, experimental settings testing anti-influenza therapies should consider this time-to-treatment; otherwise, they may provide a biased impression of effectiveness. To help translating our findings in humans, we performed an ancillary study of a prospective clinical trial and aimed to determine the symptom-to-hospitalization time in patients with community acquired pneumonia (CAP) due to influenza infection. We studied 153 patients with CAP: 37% had viral pneumonia, 24% had bacterial pneumonia, 20% had a co-infection and 19% had no identified respiratory pathogen. The most frequent identified viruses were Influenzae A virus, Rhinovirus/Enterovirus and Influenzae B virus with respectively 33%, 11% and 10% of the identified viruses. The most frequent identified bacteria were Streptococcus pneumoniae, Staphylococcus aureus, Legionella pneumophila with respectively 50%, 16% and 12% of the identified bacteria. Characteristics of patients with CAP (and with a special focus on influenza) are presented in FIG. 7 and Table 1 below:

TABLE 1
Patients' characteristics. Quantitative data are
reported as the median value and interquartile range
(IQR) and qualitative value are reported as n (%).

| | CAP (all pathogens) | | CAP (Influenza) | |
|---|---|---|---|---|
| | (n = 153) | | (n = 31) | |
| Sex (male) [n (%)] | 108 | (70.6) | 18 | (58.1) |
| Age (years) | 62 | [51-73] | 60 | [48-66] |
| BMI (kg/m²) | 27 | [23-32] | 29.5 | [27-35.5] |
| COPD [n (%)] | 108 | (24.2) | 3 | (9.7) |
| Asthma [n (%)] | 9 | (5.9) | 3 | (9.7) |
| Chronic heart | 22 | (14.4) | 8 | (25.8) |
| failure [n (%)] | | | | |
| Chronic renal | 12 | (7.8) | 4 | (12.9) |
| failure [n (%)] | | | | |
| Diabetes mellitus [n (%)] | 27 | (17.6) | 5 | (16.1) |
| Smoking status Current | 57 | (37.2) | 13 | (41.9) |
| smoker [n (%)] | | | | |
| Alcohol dependence [n (%)] | 29 | (18.9) | 4 | (12.9) |
| Immunosuppression [n (%)]¹ | 41 | (26.8) | 6 | (19.4) |

¹defined as solid cancer, hemopathy, organ transplant, bone marrow transplant, HIV infection, and splenectomy

The three main clinical symptoms reported for CAP due to influenza (A or B) infection were fever, cough and dyspnea as illustrated in FIG. 7, and Table 2 below:

TABLE 2
Symptoms and clinical signs. Quantitative data are
reported as the median value and interquartile range
(IQR) and qualitative value are reported as n (%).

| | CAP (all pathogens) | | CAP (Influenza) | |
|---|---|---|---|---|
| | (n = 153) | | (n = 31) | |
| Cough [n (%)] | 133 | (86.9) | 28 | (90.3) |
| Expectoration [n (%)] | 59 | (38.6) | 9 | (29) |
| Dyspnea [n (%)] | 140 | (91.5) | 29 | (93.5) |
| Respiratory rate (IQR/min) | 32 | [26-38] | 31 | [25-37.5] |
| Thoracic pain [n (%)] | 35 | (22.9) | 5 | (16.1) |
| Fever [n (%)] | 115 | (75.2) | 27 | (87.1) |
| Chills [n (%)] | 60 | (39.2) | 12 | (38.7) |
| Arthralgia/Myalgia [n (%)] | 35 | (22.9) | 10 | (32.3) |
| Hemoptysis [n (%)] | 12 | (7.8) | 1 | (3.2) |
| Diarrhea [n (%)] | 30 | (19.6) | 2 | (6.5) |

The symptom-to-hospitalization were 3 [2-7] days for CAP in general and 4 [3-6] days for CAP due to influenza (A or B) infection. As a result, evaluating the efficacy of an anti-influenza drug by testing it at 4 days post-infection seems to be the most pertinent approach.

Consequently, we examined the impact of administering cis-aconitate on day 4 post-infection in mice with IAV. By this stage, IAV infection had advanced to pneumonia, characterized by the substantial production of inflammatory mediators (FIG. 5b) and severe conditions manifested by ~20% body weight loss (FIG. 6d). To better emulate potential clinical translation, we administered a second dose of cis-aconitate on day 5 post-infection. In contrast to oseltamivir, cis-aconitate demonstrated significant efficacy in treating IAV-infected mice, resulting in a remarkable increase in the survival rate from 0% to 50% (FIG. 6b). Overall, we showed that critically ill mice receiving cis-aconitate in a curative manner are more efficiently treated against IAV pneumonia compared to those treated with currently recommended antivirals. Importantly, the protective benefits of cis-aconitate extend to instances where the treatment is administered within a clinically relevant timeframe, further emphasizing its potential as a promising therapeutic option for influenza infection.

### Supplementary Data

### Confirmation of the Protective Effects of Cis-Aconitate in IAV-Infected Balb/c Mice.

To validate the findings observed in C57Bl/6 mice (refer to FIGS. 6 a-d) within a different mouse species, we conducted an experiment in Balb/c mice. These (NF-kB transgenic) Balb/c mice were infected with IAV (300 PFU) and treated 2 days pi with 0.6 mg of cis-aconitate (CA) intranasally. Animal survival (FIG. 12a) and body weight loss (FIG. 12b) were monitored daily. IAV-infected, untreated Balb/c mice exhibited a complete lack of survival, contrasting sharply with a remarkable 90% survival rate observed in mice treated with cis-aconitate. The pronounced protective efficacy of cis-aconitate was further underscored by its evident impact on mitigating weight loss of IAV-infected animals. These findings highlight the substantial therapeutic potential of cis-aconitate in enhancing survival and ameliorating weight loss across different mouse strains.

Anti-Influenza Activity of Cis-Aconitate is Independent from Itaconate.

Cis-aconitate decarboxylase (CAD), also known as ACOD1 or Irg1, facilitates the conversion of cis-aconitate into itaconic acid—a key player in linking the innate immune response and cell metabolism, particularly in the context of IAV infection [18, 18, 26]. To ascertain that the observed anti-influenza effect of cis-aconitate in our mouse model of IAV pneumonia is not solely attributed to itaconate, we conducted experiments using both wild-type (WT) and CAD-deficient (CAD−/−) mice. These mice were intranasally infected with IAV and either treated with cis-aconitate or PBS (as a vehicle control), with daily monitoring of animal survival. As depicted in FIG. 13, survival patterns in CAD−/−mice closely mirrored those in wild-type mice. Consequently, these findings strongly indicate that the anti-influenza properties of cis-aconitate operate independently of itaconate.

### Example 2: Cis-Aconitate has Anti-Inflammatory Properties Potent Enough to Interrupt the Inflammatory Cascades in Play During SARS-CoV-2 Infection

### 2.1/Methods

Viruses. SARS-CoV-2 stains (Wuhan and Delta) were isolated from patients at the Academic Hospital of Tours.

Cell culture. In vitro experiments were performed using Vero Cell line or Human primary bronchial epithelial cells. Methods for generating Human primary bronchial epithelial cells culture were described in Example 1.

### 2.2. Results

To gain insight into the cis-aconitate anti-inflammatory properties during COVID-19, we infected various models of epithelial cells with two strains of SARS-CoV-2 (Wuhan and Delta, FIG. 10a). These infections induced an important release of inflammatory cytokines, exemplified here by IL-6. Remarkably, cis-aconitate treatment led to a significant reduction in IL-6 production whatever the epithelial cell model or SARS-CoV-2 strains used (FIG. 10b, 10c).

These results suggest that cis-aconitate has anti-inflammatory properties capable of disrupting the inflammatory cascades triggered during SARS-CoV-2 infection.

### REFERENCES

1. Mizgerd J P (2006) Lung infection—a public health priority. PLoS Med 3:e76. https://doi.org/10.1371/journal.pmed.0030076
2. Aliberti S, Dela Cruz C S, Amati F, et al (2021) Community-acquired pneumonia. Lancet 398:906-919. https://doi.org/10.1016/S0140-6736 (21) 00630-9
3. Guillon A, Aymeric S, Gaudy-Graffin C, et al (2017) Impact on the medical decision-making process of multiplex PCR assay for respiratory pathogens. Epidemiol Infect 145:2766-2769. https://doi.org/10.1017/S0950268817001029
4. Jain S, Self W H, Wunderink R G, et al (2015) Community-Acquired Pneumonia Requiring Hospitalization among U.S. Adults. N Engl J Med 373:415-427. https://doi.org/10.1056/NEJMoa1500245
5. Centers for Disease Control and Prevention, National Center for Immunization and Respiratory Diseases (NCIRD) (2021) Influenza (Flu) https://www.cdc.gov/ncird/flu.html
6. Influenza (Seasonal). https://www.who.int/news-room/fact-sheets/detail/influenza-(seasonal). Accessed 15 Aug. 2019
7. WHO | WHO guidelines for pharmacological management of pandemic (H1N1) 2009 influenza and other influenza viruses. In: WHO. http://www.who.int/csr/resources/publications/swineflu/h1n1_use_antivirals_20090820/en/. Accessed 15 Aug. 2019
8. Jefferson T, Jones M A, Doshi P, et al (2014) Neuraminidase inhibitors for preventing and treating influenza in healthy adults and children. Cochrane Database Syst Rev CD008965. https://doi.org/10.1002/14651858.CD008965.pub4
9. Herold S, Becker C, Ridge K M, Budinger G R S (2015) Influenza virus-induced lung injury: pathogenesis and implications for treatment. Eur Respir J 45:1463-1478. https://doi.org/10.1183/09031936.00186214
10. Tavares L P, Teixeira M M, Garcia C C (2017) The inflammatory response triggered by Influenza virus: a two edged sword. Inflamm Res 66:283-302. https://doi.org/10.1007/s00011-016-0996-0
11. Borgeling Y, Schmolke M, Viemann D, et al (2014) Inhibition of p38 Mitogen-activated Protein Kinase Impairs Influenza Virus-induced Primary and Secondary Host Gene Responses and Protects Mice from H5N1 Lethal Infection. J Biol Chem 289:13-27. https://doi.org/10.1074/jbc.M113.469239
12. Ludwig S, Wolff T, Ehrhardt C, et al (2004) MEK inhibition impairs influenza B virus propagation without emergence of resistant variants. FEBS Letters 561:37-43. https://doi.org/10.1016/S0014-5793 (04) 00108-5
13. Ludwig S (2009) Targeting cell signalling pathways to fight the flu: towards a paradigm change in anti-influenza therapy. Journal of Antimicrobial Chemotherapy 64:1-4. https://doi.org/10.1093/jac/dkp161
14. Zheng B-J, Chan K-W, Lin Y-P, et al (2008) Delayed antiviral plus immunomodulator treatment still reduces mortality in mice infected by high inoculum of influenza A/H5N1 virus. Proceedings of the National Academy of Sciences 105:8091-8096. https://doi.org/10.1073/pnas.0711942105
15. Benmoussa K, Garaude J, Acin-Perez R (2018) How Mitochondrial Metabolism Contributes to Macrophage Phenotype and Functions. J Mol Biol 430:3906-3921. https://doi.org/10.1016/j.jmb.2018.07.003
16. Stunault M I, Bories G, Guinamard R R, Ivanov S (2018) Metabolism Plays a Key Role during Macrophage Activation. Mediators Inflamm 2018:2426138. https://doi.org/10.1155/2018/2426138
17. Guillon A, Arafa E I, Barker K A, et al (2020) Pneumonia recovery reprograms the alveolar macrophage pool. JCI Insight 5:133042. https://doi.org/10.1172/jci.insight. 133042
18. O'Neill L A J, Kishton R J, Rathmell J (2016) A guide to immunometabolism for immunologists. Nat Rev Immunol 16:553-565. https://doi.org/10.1038/nri.2016.70
19. Pålsson-McDermott E M, O'Neill L A J (2020) Targeting immunometabolism as an anti-inflammatory strategy. Cell Res 30:300-314. https://doi.org/10.1038/s41422-020-0291-z
20. Guillon A, Brea-Diakite D, Cezard A, et al (2022) Host succinate inhibits influenza virus infection through succinylation and nuclear retention of the viral nucleoprotein. The EMBO Journal 41: https://doi.org/10.15252/embj.2021108306
21. Martínez-Reyes I, Chandel N S (2020) Mitochondrial TCA cycle metabolites control physiology and disease. Nat Commun 11:102. https://doi.org/10.1038/s41467-019-13668-3
22. Soto-Heredero G, Gómez de las Heras M M, Gabandé-Rodríguez E, et al (2020) Glycolysis FEBS—a key player in the inflammatory response. J 287:3350-3369. https://doi.org/10.1111/febs.15327
23. Cezard A, Guillon A, Si-Tahar M (2021) [Identification of a metabolic immune regulator in the host that protects against influenzal pneumonia]. Rev Mal Respir 38:567-571. https://doi.org/10.1016/j.rmr.2021.05.003
24. Pearce E J, Pearce E L (2018) Immunometabolism in 2017: Driving immunity: all roads lead to metabolism. Nat Rev Immunol 18:81-82. https://doi.org/10.1038/nri.2017.139
25. Rambold A S, Pearce E L (2018) Mitochondrial Dynamics at the Interface of Immune Cell Metabolism and Function. Trends Immunol 39:6-18. https://doi.org/10.1016/j.it.2017.08.006
26. Cezard A, Monard S, Brea-Diakite D, et al (2021) Les métabokines, des médiateurs essentiels de l'immunité Med anti-infectieuse. Sci (Paris) 37:342-348. https://doi.org/10.1051/medsci/2021031
27. Rao M, Dodoo E, Zumla A, Maeurer M (2019) Immunometabolism and Pulmonary Infections: Implications for Protective Immune Responses and Host-Directed Therapies. Front Microbiol 10:962. https://doi.org/10.3389/fmicb.2019.00962
28. Meischel T, Villalon-Letelier F, Saunders P M, et al (2020) Influenza A virus interactions with macrophages: Lessons from epithelial cells. Cellular Microbiology 22: https://doi.org/10.1111/cmi.13170
29. Bahadoran A, Bezavada L, Smallwood H S (2020) Fueling influenza and the immune response: Implications for metabolic reprogramming during influenza infection and immunometabolism. Immunol Rev 295:140-166. https://doi.org/10.1111/imr.12851
30. Smallwood H S, Duan S, Morfouace M, et al (2017) Targeting Metabolic Reprogramming by Influenza Infection for Therapeutic Intervention. Cell Rep 19:1640-1653. https://doi.org/10.1016/j.celrep.2017.04.039
31. Nolan K E, Baer L A, Karekar P, et al (2021) Metabolic shifts modulate lung injury caused by infection with H1N1 influenza A virus. Virology 559:111-119. https://doi.org/10.1016/j.virol.2021.03.008
32. Tian X, Zhang K, Min J, et al (2019) Metabolomic Analysis of Influenza A Virus A/WSN/1933 (H1N1) Infected A549 Cells during First Cycle of Viral Replication. Viruses 11:1007. https://doi.org/10.3390/v11111007
33. Yoshimura A, Kuroda K, Kawasaki K, et al (1982) Infectious cell entry mechanism of influenza virus. J Virol 43:284-293. https://doi.org/10.1128/JVI.43.1.284-293.1982
34. Dou D, Revol R, Ostbye H, et al (2018) Influenza A Virus Cell Entry, Replication, Virion Assembly and Movement. Front Immunol 9:1581. https://doi.org/10.3389/fimmu.2018.01581
35. Chow E J, Davis C T, Abd Elal A I, et al (2018) Update: Influenza Activity—United States and Worldwide, May 20-Oct. 13, 2018. MMWR Morb Mortal Wkly Rep 67:1178-1185. https://doi.org/10.15585/mmwr.mm6742a3
36. Le Goffic R, Balloy V, Lagranderie M, et al (2006) Detrimental contribution of the Toll-like receptor (TLR) 3 to influenza A virus-induced acute pneumonia. PLoS Pathog 2:e53. https://doi.org/10.1371/journal.ppat.0020053
37. Simmons C, Farrar J (2008) Insights into inflammation and influenza. N Engl J Med 359:1621-1623. https://doi.org/10.1056/NEJMcibr0805865
38. Blanc F, Furio L, Moisy D, et al (2016) Targeting host calpain proteases decreases influenza A virus infection. Am J Physiol Lung Cell Mol Physiol 310: L689-699. https://doi.org/10.1152/ajplung.00314.2015
39. Dai X, Zhang L, Hong T (2011) Host cellular signaling induced by influenza virus. Sci China Life Sci 54:68-74. https://doi.org/10.1007/s11427-010-4116-z
40. Gaur P, Munjhal A, Lal S K (2011) Influenza virus and cell signaling pathways. Med Sci Monit 17: RA148-RA154. https://doi.org/10.12659/MSM.881801
41. Yu J, Sun X, Goie J Y G, Zhang Y (2020) Regulation of Host Immune Responses against Influenza A Virus Infection by Mitogen-Activated Protein Kinases (MAPKs).

Microorganisms: 1067. https://doi.org/10.3390/microorganisms8071067
42. Guillot L, Le Goffic R, Bloch S, et al (2005) Involvement of toll-like receptor 3 in the immune response of lung epithelial cells to double-stranded RNA and influenza A virus. J Biol Chem 280:5571-5580. https://doi.org/10.1074/jbc.M410592200
43. Ji J, Hedelin A, Malmlof M, et al (2017) Development of Combining of Human Bronchial Mucosa Models with XposeALIR for Exposure of Air Pollution Nanoparticles. PLoS ONE 12:e0170428. https://doi.org/10.1371/journal.pone.0170428
44. Ji, Jie (2018) DEVELOPMENT OF MULTI-CELLULAR HUMAN LUNG MODELS TO STUDY INFLAMMATORY MECHANISMS AND CELL-CELL INTERACTIONS. Institutet för miljömedicin/Institute of Environmental Medicine
45. Villenave R, Thavagnanam S, Sarlang S, et al (2012) In vitro modeling of respiratory syncytial virus infection of pediatric bronchial epithelium, the primary target of infection in vivo. Proceedings of the National Academy of Sciences 109:5040-5045. https://doi.org/10.1073/pnas.1110203109
46. Tripp R A (2013) Modeling Respiratory Syncytial Virus Cytopathogenesis in the Human Airway. Am J Respir Crit Care Med 188:766-767. https://doi.org/10.1164/rccm.201308-1491ED
47. Wu N-H, Yang W, Beineke A, et al (2016) The differentiated airway epithelium infected by influenza viruses maintains the barrier function despite a dramatic loss of ciliated cells. Sci Rep 6:39668. https://doi.org/10.1038/srep39668
48. Moheimani F, Koops J, Williams T, et al (2018) Influenza A virus infection dysregulates the expression of microRNA-22 and its targets; CD147 and HDAC4, in epithelium of asthmatics. Respir Res 19:145. https://doi.org/10.1186/s12931-018-0851-7
49. Alexopoulou L, Holt A C, Medzhitov R, Flavell R A (2001) Recognition of double-stranded RNA and activation of NF-κB by Toll-like receptor 3. Nature 413:732-738. https://doi.org/10.1038/35099560
50. Yu Y-RA, O'Koren E G, Hotten D F, et al (2016) A Protocol for the Comprehensive Flow Cytometric Analysis of Immune Cells in Normal and Inflamed Murine Non-Lymphoid Tissues. PLoS ONE 11:e0150606. https://doi.org/10.1371/journal.pone.0150606
51. Perrone L A, Plowden J K, Garcia-Sastre A, et al (2008) H5N1 and 1918 Pandemic Influenza Virus Infection Results in Early and Excessive Infiltration of Macrophages and Neutrophils in the Lungs of Mice. PLoS Pathog 4:e1000115. https://doi.org/10.1371/journal.ppat. 1000115
52. Lambert Emo K, Hyun Y, Reilly E, et al (2016) Live Imaging of Influenza Infection of the Trachea Reveals Dynamic Regulation of CD8+ T Cell Motility by Antigen. PLoS Pathog 12:e1005881. https://doi.org/10.1371/journal.ppat. 1005881
53. Lim K, Kim T, Trzeciak A, et al (2020) In situ neutrophil efferocytosis shapes T cell immunity to influenza infection. Nat Immunol 21:1046-1057. https://doi.org/10.1038/s41590-020-0746-x
54. Frank K, Paust S (2020) Dynamic Natural Killer Cell and T Cell Responses to Influenza Infection. Front Cell Infect Microbiol 10:425. https://doi.org/10.3389/fcimb.2020.00425
55. Tang B M, Shojaei M, Teoh S, et al (2019) Neutrophils-related host factors associated with severe disease and fatality in patients with influenza infection. Nat Commun 10:3422. https://doi.org/10.1038/s41467-019-11249-y
56. Brandes M, Klauschen F, Kuchen S, Germain R N (2013) A Systems Analysis Identifies a Feedforward Inflammatory Circuit Leading to Lethal Influenza Infection. Cell 154:197-212. https://doi.org/10.1016/j.cell.2013.06.013
57. Shi L-N, Zhou Y, Wu C, et al (2021) LIGHT of pulmonary NKT cells annihilates tissue protective alveolar macrophages in augmenting severe influenza pneumonia. Science Bulletin S2095927321000621. https://doi.org/10.1016/j.scib.2021.01.026
58. Zhu L, Liu L, Zhang Y, et al (2018) High Level of Neutrophil Extracellular Traps Correlates With Poor Prognosis of Severe Influenza A Infection. The Journal of Infectious Diseases 217:428-437. https://doi.org/10.1093/infdis/jix475
59. Johansson C, Kirsebom F C M (2021) Neutrophils in respiratory viral infections. Mucosal Immunol 14:815-827. https://doi.org/10.1038/s41385-021-00397-4
60. Davidson S (2018) Treating Influenza Infection, From Now and Into the Future. Front Immunol 9:1946. https://doi.org/10.3389/fimmu.2018.01946
61. Lhommet C, Garot D, Grammatico-Guillon L, et al (2020). Predicting the microbial cause of community-acquired pneumonia: can physicians or a data-driven method differentiate viral from bacterial pneumonia at patient presentation? BMC Pulm Med March 6; 20 (1): 62. doi: 10.1186/s12890-020-1089-y. PMID: 32143620; PMCID: PMC7060632.
62. Ferreira A, Sacramento C, Pereira-Dutra F, et al. (2023). Severe influenza infection is associated with inflammatory programmed cell death in infected macrophages. Front Cell Infect Microbiol. 2023 Feb. 16:13:1067285. doi: 10.3389/fcimb.2023.1067285. eCollection 2023.
63. Matute-Bello G, Frevert C, Martin T (2008). Animal models of acute lung injury. Am. J. Physiol. Lung Cell Mol. Physiol. 2008; 295: L379-399. doi: 10.1152/ajplung.00010.2008.

1. A method of treatment of a viral lung infection and/or of an adverse immune response to a viral lung infection in a subject in need thereof, comprising administering to the subject an effective amount of aconitate or pharmaceutically acceptable salt thereof.

2. The method according to claim 1, wherein the aconitate is cis-aconitate or trans-aconitate.

3. The method according to claim 1, wherein the aconitate has immunomodulatory properties and/or antiviral properties, wherein:
the immunomodulatory properties of aconitate include the reduction or inhibition of the inflammation response to the viral lung infection;
the antiviral properties of aconitate include the inhibition of viral replication.

4. The method according to claim 1, wherein the viral lung infection is caused by a respiratory virus preferably selected from an influenza virus, a coronavirus, a respiratory syncytial virus, a parainfluenza virus, a metapneumovirus, a rhinovirus, an adenovirus, a varicella-zoster virus, a cytomegalovirus, a paramyxovirus such as Nipah virus, and a bocavirus.

5. The method according to claim 1, wherein the viral lung infection is caused by an influenza virus or a coronavirus.

6. The method according to claim 1, wherein the method is applied to treat a viral lung infection.

7. The method according to claim 1, wherein the method is applied to treat an adverse immune response to a viral lung infection in a subject with a lung viral infection.

8. The method according to claim 1, wherein the method is applied to prevent a lung viral infection from escalating to an adverse immune response in a subject with a lung viral infection.

9. The method according to claim 1, wherein the adverse immune response to viral lung infection is the adverse immune response to the lung viral infection is a hyper-inflammatory immune response, a dyspnea, a tachypnea, a pneumonia in particular an acute pneumonia, an exacerbation of a chronic respiratory disease such as asthma or chronic obstructive pulmonary disease, a sepsis, a septic shock, a cytokine storm or an acute respiratory distress syndrome (ARDS).

10. The method according to claim 1, wherein aconitate is administered between 4 and 14 days post infection.

11. The method according to claim 1, wherein the subject is an animal, preferably a human, a domestic bird or a pig.

12. The method according to claim 1, wherein aconitate is used alone or in combination with one or more active substance selected from the group consisting of antivirals, antibiotics, and/or antalgics.

13. The method according to claim 1, wherein said treatment is for preventing the occurrence of a lung viral infection, the method comprising administering aconitate in a subject that is not infected with a respiratory virus.

14. The method according to claim 1, wherein the composition is administered intrapulmonary, nasally, orally, enterally, intravenously, intramuscularly and subcutaneously.

15. The method according to claim 1, comprising administering a pharmaceutical composition comprising aconitate or a pharmaceutically acceptable salt thereof and a pharmaceutically acceptable support.
